(19) United States
(12) Patent Application Publication
(10) Pub. No.: US 2011/0275643 A1
(43) Pub. Date: Nov. 10, 2011

(54) AROYLQUINOLINE COMPOUNDS

(75) Inventors: Jing-Ping Liou, Taipei City (TW); Jang-Yang Chang, Taipei City (TW)

(73) Assignees: NATIONAL HEALTH RESEARCH INSTITUTES, Miaoli County (TW); TAIPEI MEDICAL UNIVERSITY, Taipei City (TW)

(21) Appl. No.: 12/912,260

(22) Filed: Oct. 26, 2010

(30) Foreign Application Priority Data
May 6, 2010 (TW) 099114567

(51) Int. Cl. A61K 31/47; C07D 241/42; C07D 239/72; C07D 215/38; A61P 35/00; C07D 215/20; C07D 401/04; A61K 31/498; A61K 31/517; A61K 31/4709; C07D 215/48; C07D 215/36

(52) U.S. Cl. 514/249; 546/168; 544/353; 546/157; 544/283; 546/160; 546/178; 546/167; 514/311; 514/312; 514/266.1; 514/313; 514/314

(57) ABSTRACT

A serious of nitro heterocyclic derivatives including a structure of formula (I) are provided. In formula (I), P, Q and R1 to R8 are defined in the specification. The derivatives disclosed in the present invention are characterized in inhibiting tubulin polymerization, and treating cancers and other tubulin polymerization-related disorders with a suitable pharmaceutical acceptable carrier.

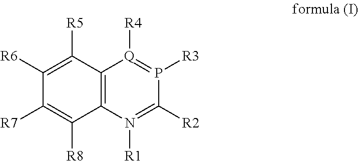

### FIELD OF THE INVENTION

The present invention relates to a series of nitro heterocyclic derivatives for treating cancer cells and the preparation method therefor. In particular, the present invention relates to a series of nitro heterocyclic pharmaceutical compositions with medicinal effect by inhibiting microtubule activity of cancer cells and the preparation method therefor.

### BACKGROUND OF THE INVENTION

At present, tubulin polymerization inhibitor should be one of the most effective anticancer drugs in the clinical application. The anticancer effect usually is performed via the tubulin depolymerization or stabilization. Microtubule is an important component for mitosis in the cells and relates to cellular migration, adhesion and intracellular transportation. Vinca alkaloids derivatives, especially vincristine and vinblastine, have been used in clinics for many years. Recently, Navelbine is found to be used in treating breast adenocarcinoma, and the semi-synthetic new drug, vinflunine, also enters into the clinical development stage. Fukada, T. (2007) indicates that such drugs belong to anti-mitotic agent and are able to arrest the mitosis assembly. In addition, another clinically active drug, paclitaxel, exhibits the anticancer activity by promoting the stable formation of non-functional microtubule, and thus the drugs with absolutely different mechanism are used to interfere microtubule and represent the effective therapeutic effect. Recently, a naturally multi-hydroxy debenzyl ethane compound, combretastatine A-4 (abbreviated as CA4, referring to compound 1 in FIG. 1(a)), which is extracted from Combretum caffrum's bark, is concerned. In difference with the traditional compound that is directly functioned on cancer cells, CA4 functions on the blood vessels of tumor to induce abundantly morphological changes of endothelial cells so that tumor capillaries are blocked. Pharmacological experiment indicates that CA4 is able to function in tumor capillary system in many substantial tumor model and shut down the blood supply to tumor. Since the amount of CA4 is few in the natural plants, it is not found that any plant contains CA4 in Taiwan. At the same time, since CA4 has low solubility, it results in shortage in supply and is difficult to show the pharmacological effect/function sufficiently. Resolving the problems on CA4 supply and solubility becomes one of the focused researches to many pharmacologists in recent years. The representative drug for increasing solubility is combrestatine-A4 phosphate (CA4P, compound 2, as shown in FIG. 1(a)), which is a prodrug of combretastatin A-4 disodium phosphate ester. Siemann et al. (2009) considers that CA4P significantly increases the anti-tumor activity. Colchicine (compound 3, referring to FIG. 1(c)) is a toxin relevant to mitosis, wherein the ring-C of colchicines is conjugated with microtubule to disrupt microtubule to polymerize as spindle fibers and arrest mitosis at M phase. Colchicine is able to inhibit bone marrow to reduce the numbers of leukocytes and platelets. Colchicine has higher toxicity and might damage the gastrointestinal tract, central nervous, circulation system, hematopoietic system and kidney. However, adverse drug reaction is absent at appropriately small dosage (such as 0.5 mg, twice per day) or even the long term administration. As Mauer et al.'s research (2008) at phase II, it indicates that an anti-microtubule agent, ABT-751 (compound 4, referring to FIG. 1(c)), functions at the M phase of cell cycle and the main function of ABT-751 and tubulin polymerization have anti-angiogenesis activity at the same time.

The current available clinically used chemotherapeutic microtubule inhibitors have high toxicity, and their potential is limited by the development of multidrug resistance (MDR). Therefore, there has been great interest in identifying novel microtubule inhibitors that overcome various modes of resistance and exhibited improved pharmacology profiles. Quinoline is a class of compounds based on heterocyclic structure, and some quinolines have been used in cardiovascular disease and are pharmacologically active drugs. Analysis of the quinoline compounds 1, 3 and 4, indicates that such compounds represent the inhibition activity on microtubule and are relevant with the 3,4,5-trimethoxyphenyl/3,4,5-trimethoxybenzoyl and para-methoxyphenyl groups of the basic skeleton.

It is therefore attempted by the applicant to deal with the above situation encountered in the prior art.

### SUMMARY OF THE INVENTION

A serious of a nitro heterocyclic derivatives having a formula I are provided as follows:

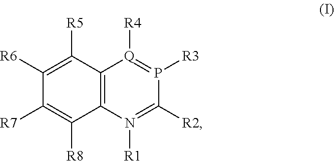

wherein P and Q respectively are (i) a first carbon and a second carbon, (ii) a first nitrogen and the second carbon or (iii) the first carbon and a second nitrogen, R1 is a first substituted group being one selected from a group consisting of null, an oxygen, a first C₁-C₈ alkoxy group, a first C₁-C₈ hydrocarbon group and a first C₁-C₈ alkyl halide group, and R2 to R8 respectively are a second substituted group to an eighth substituted group, each of which is one selected from a group consisting of a first hydrogen, a first halide group, a hydroxyl group, a first amino group, a first cyano group, a first nitro group, an aroyl group, a first disodium hydrogen phosphate group, a first diammonium hydrogen phosphate group, a first dipotassium hydrogen phosphate, a first monocalcium phosphate group, a second C₁-C₈ alkoxy group, a C₁-C₈ aromatic group, a second C₁-C₈ hydrocarbon group, a C₁-C₈ alkylthio group, a C₁-C₈ alkyl nitro group, a second C₁-C₈ alkyl halide group, a C₁-C₈ hydroxyl group, a C₁-C₈ aldehyde group, a C₁-C₈ ester group, a C₁-C₈ acidic group, a C₁-C₈ ether group and a C₁-C₈ amide group. The first C₁-C₈ alkyl halide group and the second C₁-C₈ alkyl halide group have a second halide group therein being one selected from a group consisting of a fluoride, a chloride, a bromide and an iodide. When R1 is bound with the above first substituted group, the N-R1 group forms a cationic group.

Preferably, “alkyl group” is referred to an alkyl group with a C≦10 unbranched chain, a C≦10 branched chain or a 3≦C≦5 cyclic alkyl group. Further, the alkyl group also includes the saturated hydrocarbon group and the unsaturated alkenyl group or alknyl group. The examples of the saturated hydrocarbon group include but not limit in methyl, ethyl, propyl, isopropyl, text-butyl, pentyl, iso-pentyl, n-hexyl, iso-hexyl, cyclopropyl, cyclobutyl, cyclopentyl and cyclohexyl groups. The examples of the unsaturated hydrocarbon group includes vinyl, allyl, allenyl, butenyl, butadienyl, acetenyl, propynyl, butynyl and so on.

Preferably, “aromatic group” is referred to a C≧5 cyclic group, which includes the heterocyclic group containing nitrogen (N), oxygen (O), sulfur (S) and/or phosphorus (P). If necessary, the cyclic structure of the aromatic group is bound with C₁-C₃ alkyl group, C₁-C₃ alkyl sulfur group, C₁-C₃ alkyl halide group, halide, hydrocarbon group, amino group, cyano group, nitro group and so on. The examples includes but not limit in pyrroline, furyl, thiophene, phosphole, benzyl, pyridinyl, pyranyl, thiapyran, phosphorine, methylpyridinyl, butylpyrridinyl, furyl halide, quinoline, quinazoline and quinoxaline.

Preferably, P and Q of Formula I are carbon (C), or any one of P and Q is nitrogen (N), and the nitro heterocyclic derivative can be represented as quinoline (Formula II), quinazoline (Foimula III), quinoxaline (Formula IV).

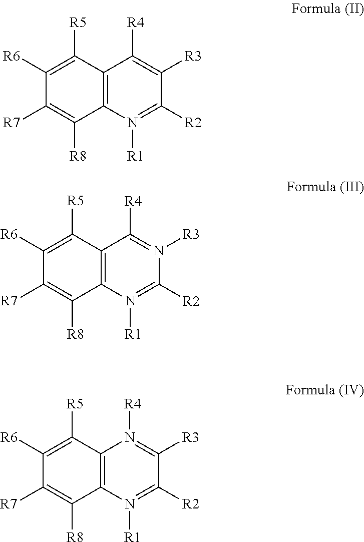

A serious of “aroyl” derivatives in the present invention are referred to that R2 to R8 substituted groups of the nitro heterocyclic compounds, quinoline, quinzaoline and quinioxaline, can be an —ArX group, a —CH₂—ArX group, an —O—ArX group, a —CO—ArX group, a —CH₂O—ArX group, a —CO—O—ArX group, an —S—ArX group, an —SO₂—ArX group, an —NH—ArX group and so on. Furthermore, the “ArX” of the above substituted groups is an aromatic group having least one X group bound thereon, and the X group can be hydrogen, halogen, amino group, cyano group, C₁-C₃ alkyl group, C₁-C₅ hydrocarbon group, C₁-C₃ alkylthio group, C₁-C₃ alkyl nitro group, C₁-C₃ amide group, C₁-C₃ hydroxyl group, C₁-C₃ alkyl halide group, disodium hydrogen phosphate group, diammonium hydrogen phosphate group, dipotassium hydrogen phosphate group or monocalcium phosphate group.

The compounds of the quinoline (Formula II), quinazoline (Formula III) and quinoxaline (Formula IV) derivatives disclosed in the embodiments of the present invention are provided as follows.

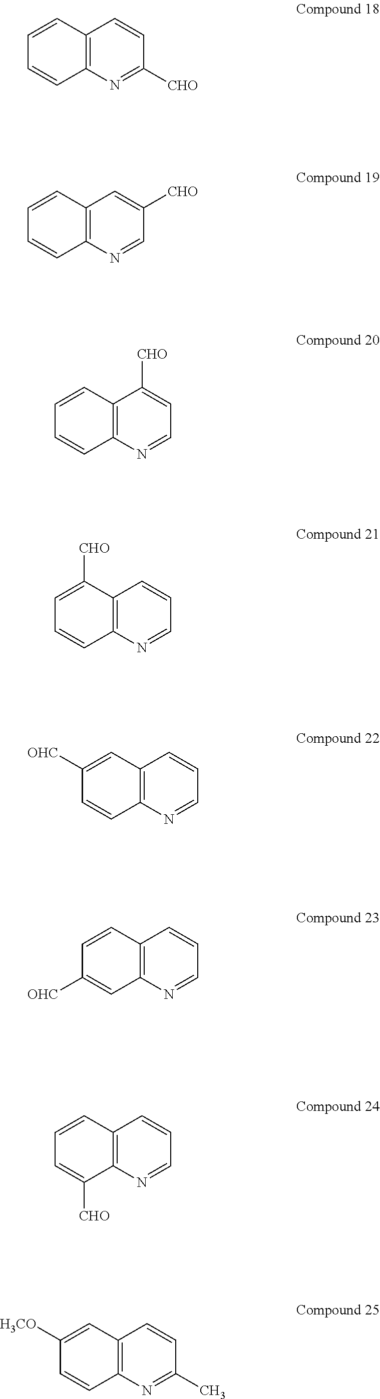

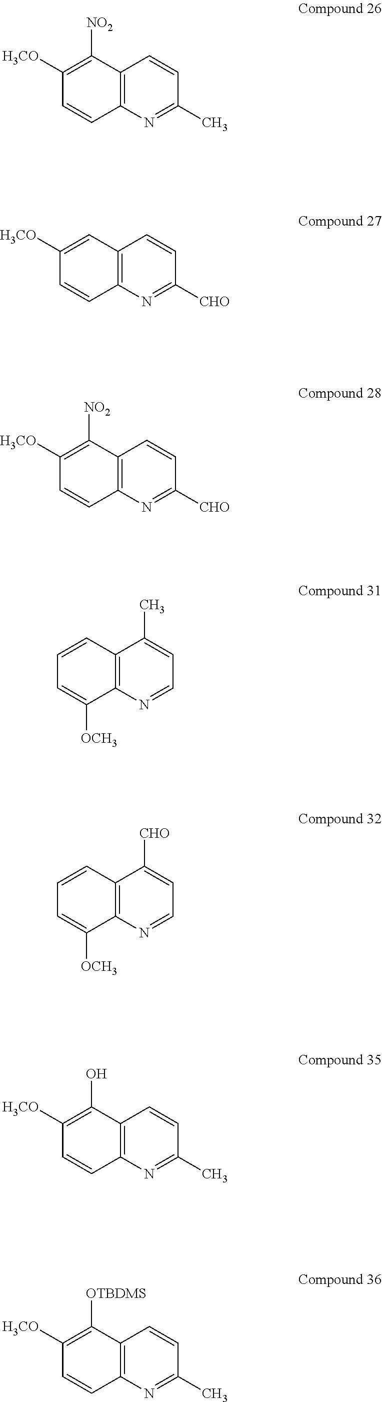

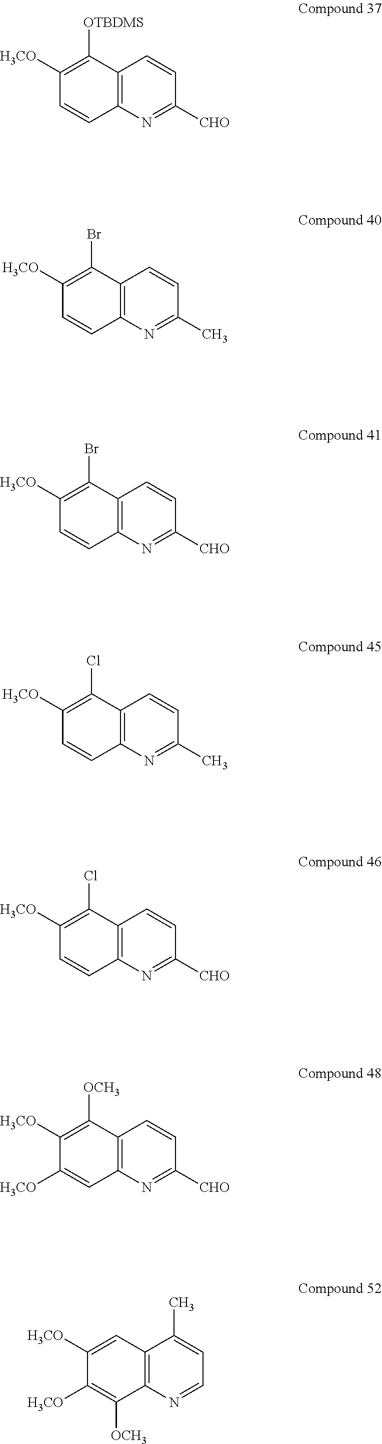

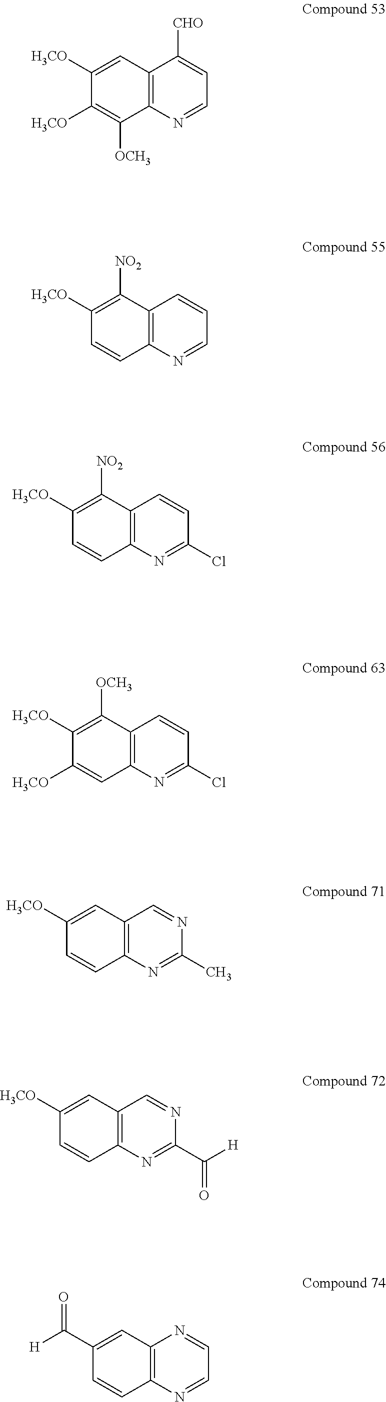

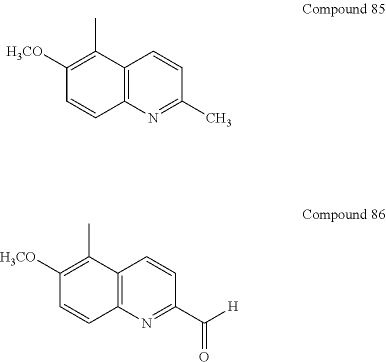

The R2 to R8 substituted groups of quinoline (Formula II), quinazoline (Formula III) and quinoxaline (Formula IV) derivatives in the present invention adopt the aroyl substituted group(s) as the aroyl derivatives, and the embodiments of the aroyl derivatives are disclosed as follows.

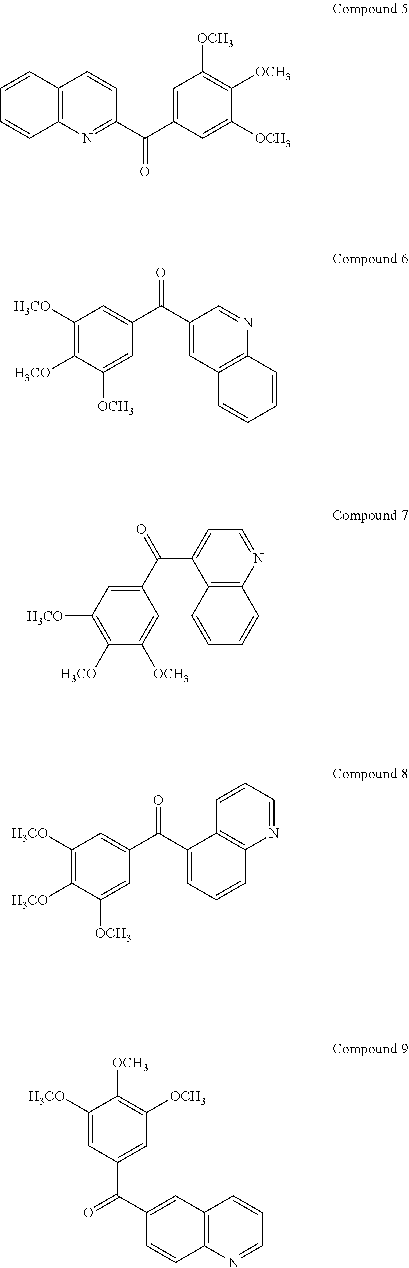

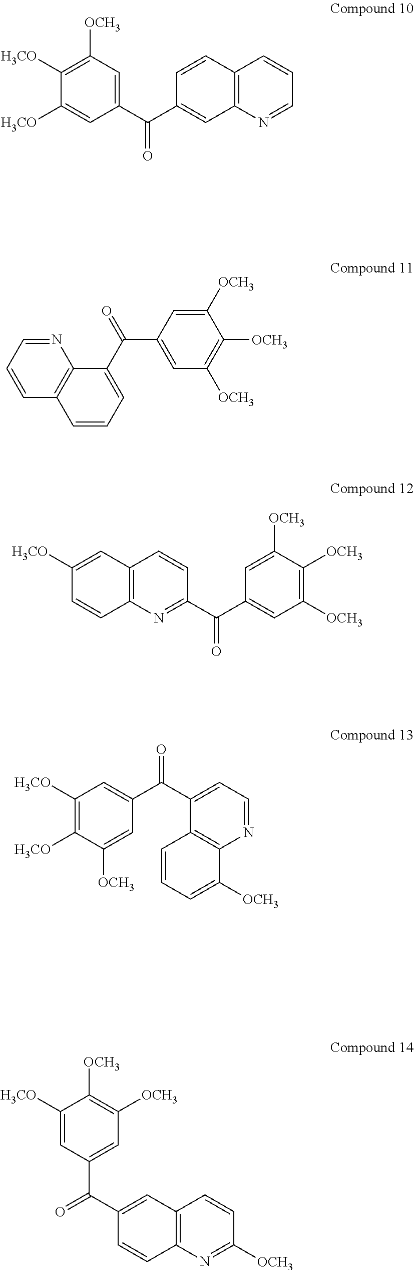

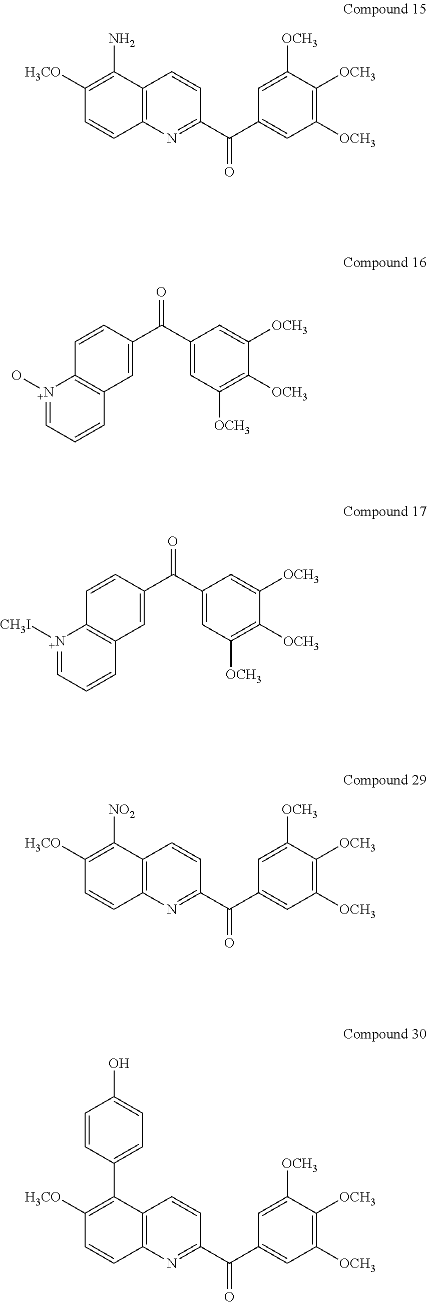

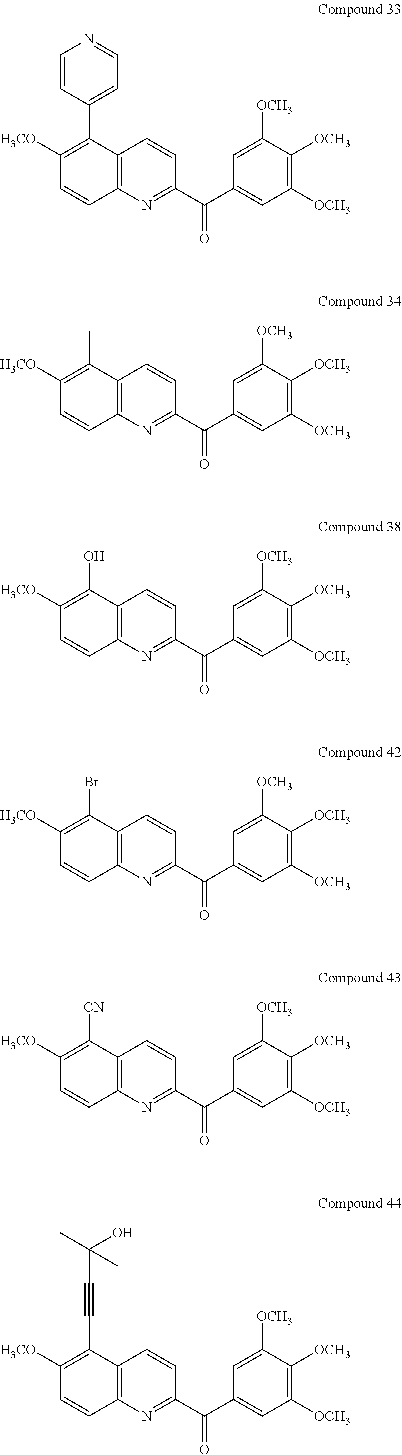

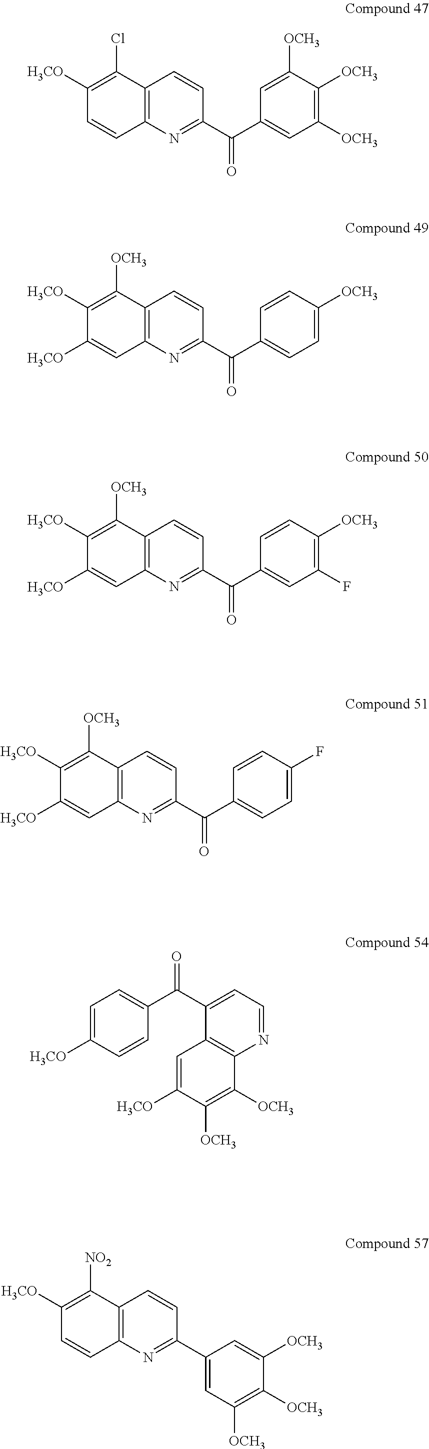

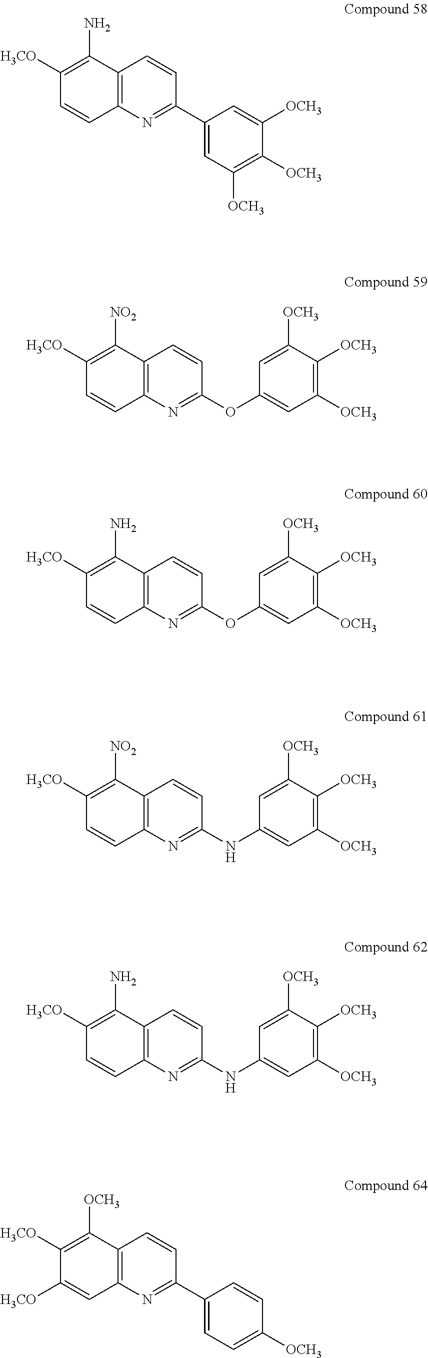

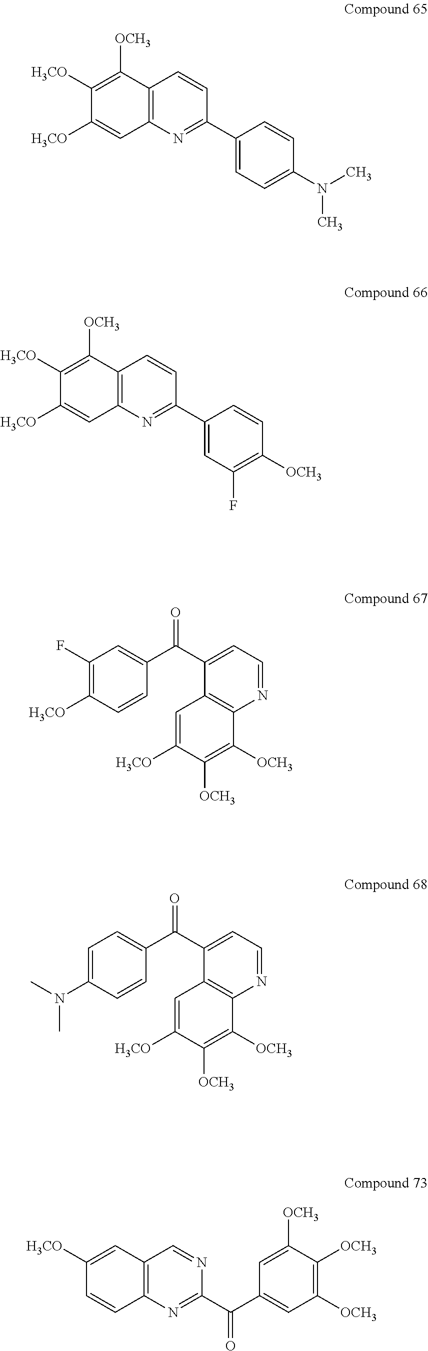

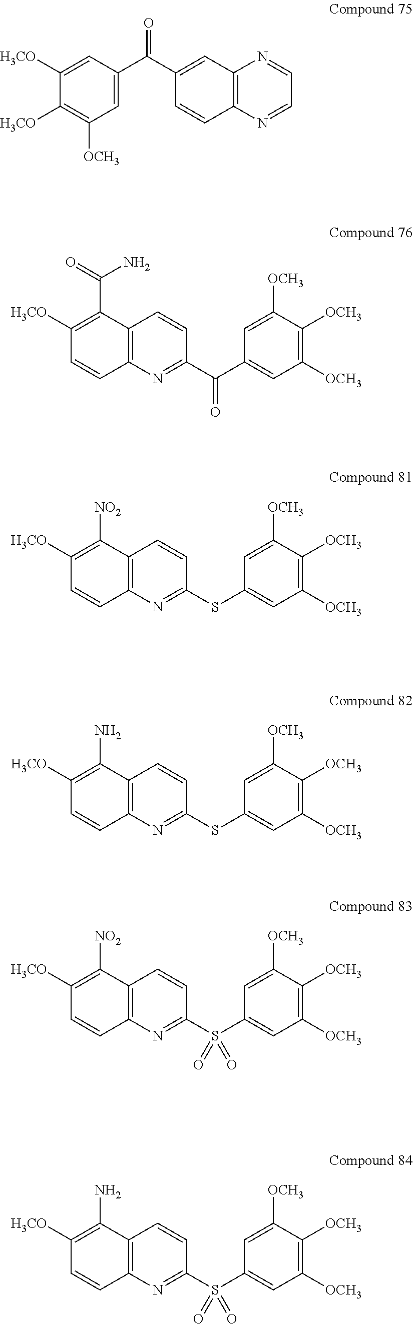

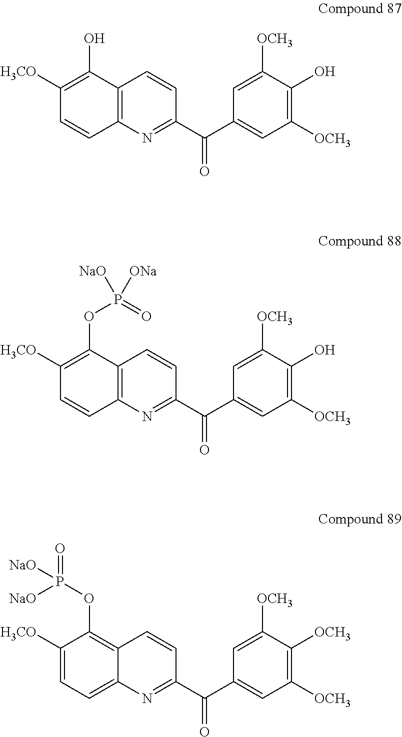

Preferably, as shown in FIG. 2, aniline compound with different substituted groups (ZR) is synthesized as a serious of quinoline derivatives (formula II). For instance, 8-methoxy-4-methylquinoline (compound 31) was afforded using o-anisidine as raw material and supplementing reagents such as ferric chloride (FeCl₃) and methyl vinyl ketone. Based on the preparation method, compound 52 with a methyl at the R4 position was afforded using 3,4,5-trimethoxyaniline as raw material. A solution of 3,4,5-trimethoxyaniline in hydrochloride (HCl) was added zinc chloride (ZnCl₂) and selenium dioxide (SeO₂) respectively to afford a quinoline compound 48 with a formyl at the R2 position. The acidic solution was added nitrobenzene, ferrous sulfate (FeSO₄·7H₂O) and glycerol to afford a quinoline compound 63 with a chloride at the R2 position. The reference numeral, “2a”, in FIG. 2 represents the reagents, SeO₂ and so on in the reaction.

2-Bromo-5-methoxybenaldehyde and cuprous iodide were mixed with dimethylformamide (DMF) to heat to afford 6-methoxy-2-methylquinazoline (compound 71) having a structure of Formula (III) and a methyl at the R2 position. Compound 71 further was reacted with xylene and SeO₂ to afford compound 72 with an aldehyde group at the R2 position. Compound 72 was reacted with phenylmagnesium bromide with different substituted groups in the Grignard reaction, and was oxidized using pyridinium dichromate (PDC) to afford an aroyl compound, 6-methoxy-2-(3′,4′,5′-trimethoxybenzoyl)quinazoline (compound 73). Since the methyl at the R2 position was substituted as an aldehyde group, the aroyl compounds having benzoyl group at the R2 position can be afforded using different reactants via the similar pathway. The synthetic pathway shown in FIG. 2(b) also can be applied in the quinoline derivatives having a structure of formula (I). For instance, compounds 25, 26, 40, 45 with a methyl at the R2 position respectively were synthesized as compounds 27, 28, 41, 46 with an aldehyde group at the R2 position, and then compounds 27, 28, 41, 46 respectively were synthesized as aroylquinoline compounds 12, 29, 42, 47 with a benzoyl group at the R2 position. The reference numerals, “2b”, “2c” and “2d”, in FIG. 2(b) are respectively referred to the reagents used in each reaction steps.

The present invention discloses that 3,4,5-trimethoxyphenyl-magnesium bromide and the raw material such as quinoline formamide, quinazoline formamide or quinoxaline formamide with different substituted groups at different positions are reacted to synthesize the diverse aroyl derivatives having structures of formulas (II), (III) and (IV). In addition to the above substitutions, compound 55 was able to react with dichloromethane (CH₂Cl₂) and benzoic acid to afford compound 56 with a chloride group at the R2 position, and then compound 56 was reacted with 3,4,5-trimethoxyaniline to afford an aroylquinoline compound 61 with a trimethoxyphenoxy group at the R2 position. Compound 56 with a chloride group at the R2 position was reacted with reagents such as tetrakis(triphenylphosphine)palladium and 3,4,5-trimethoxyphenylboronic acid, etc. to afford compound 57 with a trimethoxyphenyl group at the R2 position and a nitro group at the R5 position. Compound 57 was reacted with reagents such as isopropanol and iron powder, etc. to afford compound 58 with an amino group at the R5 position. 6-Methoxy-2-methylquinoline (compound 25) was reacted with nitric acid (HNO₃) and sulfuric acid (H₂SO₄) to synthesize compound 26 (named as 2-methyl-6-methoxy-5-nitroquinoline) having a nitro group at the R5 position.

5-Amino-6-methoxy-2-(3′,4′,5′-trimethoxybenzoyl)quinoline (compound 15) in the concentrated H₂SO₄ solution was added dropwise to a soduium nitrite (NaNO₃) solution and diazonium salt solution to afford compound 87 with a 5-hydroxy group at the R5 position. Subsequently, compound 87 was reacted with reagents such as anhydrous acetonitrile, N,N-dimethylaminopyridine, dibenzyl phosphite (DBP) and so on to afford 5-[6-methoxy-2-(4′-hydroxy-3′,5′-dimethoxybenzoyl)quinoline]disodium phosphate (compound 88).

Quinolines with methoxy groups at the R2 to R8 positions (compounds 18 to 24, i.e. 2-, 3-, 4-, 5-, 6-, 7- and 8-quinoline carboxaldehydes) were reacted with raw materials bounding with aldehyde groups and tetrahydrofuran (THF) to synthesize a serious of aroylquinoline derivatives (compounds 5 to 11) having a trimethoxybenzoyl substituted group at the R2 to R8 positions. The reference numeral “3a” in FIG. 3(a) is referred to the usage of reagents, such as THF, etc.

Based on the preparation method described in FIG. 3(a), compound 9, being raw material, was mixed with CH₂Cl₂ and m-chloroperbenzoic acid (m-CPBA), and the semi-product was extracted after reaction. The semi-product was further purified, reacted with phosphoryl chloride (POCl₃), dissolved in sodium methoxide to heat at reflux, and purified by silica gel column chromatography to afford compound 14. The reference numeral “3b” in FIG. 3(b) is referred to the usage of reagents such as CH₂Cl₂, etc.

Compounds 16 (95% yield) and 17 (91% yields), being the N1-substituted quaternary salt derivatives, were made using compound 9 as raw material. Compound 16 was made by reacting compound 9 with CH₂Cl₂ and m-CPBA, and compound 17 was prepared by reacting compound 9 with iodomethane (CH₃I). As shown in FIG. 3(b), reference numerals “3c” and “3d” are referred to the usage of reagents, m-CPBA and CH₃I, etc.

Compound 25 with a methyl at the R2 position was able to be converted as an intermediate with an aldehyde group, and then Grignard reaction was performed using 3,4,5-trimethoxyphenylmagnesium bromide and oxidation was regulated with PDC to synthesize compound 12, which has 49% yield after three steps. Compound 15 has one more amino group on quinoline structure than compound 12, and the synthesized critical intermediate is compound 26. Compound 26 was made via four steps, including oxidation of the R2 position regulated with SeO₂, Grignard reaction using 3,4,5-trimethoxyphenylmagnesium bromide, oxidation regulated with PDC and reduction with sodium sulfide (Na₂S), and thus compound 15 (24% yield) was afforded from compound 25.

Compound 13 (17% yield) was afforded from the commercial o-anisidine via a four-step reaction. In addition, methyl vinyl ketone was dissolved in acetic acid, and then ferric chloride and zinc chloride were added to afford 8-methoxy-4-methylquinoline (compound 31). If compound 31 was oxidized with a p-xylene solution containing SeO₂ to afford compound 32 with a formyl group at the R4 position, and then Grignard reaction was performed on compound 32 using 3,4,5-trimethoxyphenylmagnesium bromide and oxidation was performed with PDC, compound 13 with a 3′,4′,5′-trimethoxybenzoyl group at the R4 position was afforded. The physical properties of the above-mentioned synthesized compounds are listed in “Detailed Description of the Preferred Embodiment”.

Preferably, the compounds of the present invention are used to inhibit microtubule polymerization in the cells or inhibit the microtubule polymerization-associated cancers in the mammals, in particular to the effect of inhibition, remission, treatment and therapy on human. Except for a particular announcement, the above derivatives disclosed in the present invention are prepared as monomer, salts, solvents, prodrugs, crystals, hydrates, tautomers, disastereomers, enantiomers or metabolites to be prepared as the pharmaceutical compositions for medicinal effect with the pharmaceutically acceptable carrier or excipient.

“Salts” are the derivatives disclosed in the present invention, where salts are prepared from conjugating quinoline, quinazoline, quinoxaline or the series of aroyl derivatives having positive charge with the adequate anion. The adequate anion includes chloride, bromide, iodide, sulfate ion, bisulfate ion, sulfamate ion, nitrate ion, phosphate ion, methanesulfonate ion, trifluoroacetate ion, citrate ion, glutamate ion, glucuronate ion, glutarate ion, malate ion, maleic ion, succinate ion, fumarate ion, tartrate ion, tosylate ion, salicylate ion, naphthalenesulfonate ion, lactate ion and acetate ion. Similarly, a quaternary nitrogen salts are prepared from conjugating quinoline, quinazoline, quinoxaline or the series of aroyl derivatives having negative charge with the adequate cation. The adequate cation includes sodium ion, potassium ion, magnesium ion, calcium ion and ammonium cation such as tetramethylammonium ion.

“Prodrug” means that the derivatives disclosed in the present invention form the esters to be prepared as the adequate dosage forms with other pharmaceutically acceptable carrier or excipient. The dosage form containing prodrug can be converted as quinoline (formula (II)), quinazoline (formula (III)), quinoxaline (formula (IV)) and the aroyl derivatives in vitro or in vivo and does not eliminate the activity or property of the derivatives, or provides efficient medicinal effect without relatively increasing any toxicity. According to the demand, the hydroxyl group of quinoline, quinazoline, quinoxaline and the aroyl derivatives is conjugated with carbonate or phosphate to form the ester prodrug, which is hydrolyzed in vitro or in vivo to represent a hydroxyl group of derivative. The amino group of quinoline, quinazoline, quinoxaline or the aroyl derivative can form the prodrug having the amide, carbamate or imine.

“The pharmaceutically acceptable carrier or excipient”, or named as “the bioavailable carrier or excipient”, includes any currently adequate compound for preparing as the dosage forms, such as solvent, dispersing agent, coating, antibacterial agent or antifungal agent, preservative, absorption delay agent and so on. Such carriers or excipients usually lack activity on treating diseases. Further, each dosage form which is prepared by incorporating the derivatives disclosed in the present invention with the pharmaceutically acceptable carrier and excipient does not result in the adverse drug reaction, allergy or other inappropriate reactions upon administrating on animals or humans. Thus, the derivatives disclosed in the present invention which are incorporated with the pharmaceutically acceptable carrier or excipient are suitable in clinics and veterinary medicine. The dosage forms of compounds of the present invention are administrated via the intravenous, oral, nasal, colonal, vaginal or sublingual medication to achieve the therapeutic effect. For example, the cancer patient is administrated with the oral dosage form (about 0.1 mg to 50 mg active ingredient per day).

Carrier depends on the various dosage forms. The aseptic injection composition is made by dissolving or suspending the derivatives in the non-toxic intravenous diluent or solvent such as 1,3-butanediol. The acceptable carrier can be mannitol or water. In addition, the fixed oil or the synthetic mono- or di-glyceride for suspending the medium belongs to the regular solvent. Fatty acid, such as oleic acid, olive oil, castor oil, or the glyceride derivatives therefor, in particular to the composition via the multi-oxygen ethylation, can be prepared as the injection agent and can be the naturally and pharmaceutically acceptable oils. Such oil solutions or suspensions can include the long-chain alcohol diluent, dispersing agent, carboxymethyl cellulose or the similar dispersing agent. Other common surfactants are used such as Tween, Spans, other similar emulsifiers, or the bioavailable enhancers which are the pharmaceutically acceptable solids, liquids or others for developing dosage forms in the regular pharmaceutical industry.

The orally dosed composition adopts any one of the orally acceptable dosage forms including capsule, tablet, pill, emulsifier, suspension liquid, dispersing agent and solvent. Regarding the carrier generally used in the oral dosage form, taking tablet as the example, it can be lactose, corn starch, lubricating agent, and magnesium stearate is the basic supplement. Diluent used in capsule includes lactose and the dried corn starch. The dosage form of liquid suspension or emulsifier is made by suspending or dissolving the active material in the oil interface which combines with emulsifier or suspension, and the adequate edulcorant, flavor or pigment is supplemented on demand.

The nasal aerosol or inhalation composition can be manufactured according to the known preparation technology. For instance, the composition is dissolved in saline, and benzyl alcohol, other adequate preservative or absorbefacient is added to improve the bioavailability. The composition of the compounds in the present invention also can be prepared as suppository to be administrated via colon or vagina.

The compounds of the present invention also are used in the “intravenous administration”, which includes intradermal injection, intraperitoneal injection, intravenous injection, intramuscular injection, intra-articular injection, intracerebral injection, visco-supplementation, spinal injection, arterial injection, intrapleural injection, injection at the disease organ/tissue and other appropriate administration techniques.

“Cancer” is referred to the cells with the over-proliferative activity and also is considered as the cells that are situated at the abnormal growth condition or have rapid proliferation property. In addition, cancer cells might include P-glycoprotein (P-gp), multidrug resistance (MDR)-associated proteins, lung carcinoma drug resistance-associated protein, breast carcinoma drug-resistance protein or other drug resistance-associated proteins associated with other anticancer drugs expressed by cancer cells. The cancer indicated in the present invention includes but not limit to leukemia, sarcoma, osteosarcoma (malignant neoplasm of bone), lymphoma, melanoma, ovary cancer, epidermal carcinoma, skin cancer, testicular cancer, gastric cancer, pancreas cancer, kidney cancer, breast carcinoma, prostate cancer, colon cancer, head and neck cancer, brain tumor, esophageal cancer, bladder cancer, adrenocortical carcinoma, lung cancer, bronchial carcinoma, endometrial cancer, cervical cancer, nasopharyngeal carcinoma, liver cancer or unidentified cancer.

The analysis and identification methods for the synthesized derivatives of the present invention are listed as follows. Nuclear magnetic resonance spectra (¹H NMR) were obtained with Bruker DRX-500 spectrometer (operating at 500 MHz), with chemical shift in parts per million (ppm, δ) downfield from tetramethylsilane (TMS) as an internal standard. High-resolution mass spectra (HRMS) were measured with a JEOL (JMS-700) electron impact (EI) mass spectrometer. Flash column chromotography was down using silica gel (Merck Kieselgel 60, No. 9385, 230-400 mesh ASTM).

Evaluation of Bioactivity

(A) In Vitro Growth Inhibition of Cells

The synthesized compounds 5 to 17 were evaluated for antiproliferative activities against four carcinoma cells, oral epidermoid carcinoma KB cells, non-small-cell lung carcinoma H460 cells, colorectal carcinoma HT29 cells and stomach carcinoma MKN45 cells, as well as the MDR-positive cell lines KB-VIN10, that overexpressed P-gp 170/MDR (Table 1). The controls were compounds 1 and 3.

First, the position effect of aroyl group (3,4,5-trimethoxybenzoyl) in the quinoline system was evaluated. As listed in Table 1, the regioisomers having different substituted groups at the R2 to R8 positions were compounds 5, 6, 7, 8, 9, 10 and 11, which were evaluated for antiproliferative activity against five cancer cell lines. The 3,4,5-trimethoxybenzoyl group ring resulted in the most potent activity with compound 5 and 9 showing mean IC₅₀ value of 172.85 and 24.4 nM against five cancer cell lines, respectively. Shifting of the aroyl group to the R3, R4, R5 or R8 position resulted in weak cytotoxicity at the μM level, while shifting to the R7 position, as in compound 10, resulted in the loss of cytotoxicity.

According to Pettit et al.'s research (2003), the p-methoxy group substitution in the ring-B of cis-stilbene (compound 1) is important for activity, while Yoshino et al.'s study (1992) on ABT-751 (compound 4) was found that the p-methoxy group substitution in the 3-benzenesulfonamide of pyridine is relevant with activity. The methoxy group at the R6 position of compound 12 is at the opposite site of aroyl substituted group at the R2 position thereof, and compound 13 (8-methoxy-4-(3′,4′,5′-trimethoxybenzoyl)quinoline) and compound 14 (2-methoxy-6-(3′,4′,5′-trimethoxybenzoyDquinoline) also had the similar opposite-site relationship. All three compounds showed substantial antiproliferative activity against five cancer cell lines with mean IC₅₀ values of 67, 164, and 220 nM, respectively. Introduction of a methoxy group at the R6 and R8 positions of compound 5 and compound 7, gave compound 12 and compound 13, respectively, with increases in cell growth inhibition ability as compared to the parental compound. Compound 13 showed over an order of magnitude increase in activity over the parental compound 7, while compound 12 showed improved of IC₅₀ values to double digit nanomolar values in the KB, H460, HT29, and KB-VIN10 cell lines. However, the addition of a methoxy group at the R2 position in compound 14 resulted in a decrease in potency as compared to compound 9. In an effort to increase the 2-aroylquinolines skeleton's polarity and activity, compound 15 having an amino group at the R5 position and a methoxy group at the R6 position was synthesized. It exhibited a mean IC₅₀ value of 0.32 nM in all five cancer cell lines, thus displaying stronger cytotoxicity than compound 1. Compound 15 with an additional amino group at the R5 position showed approximately>100-fold improvement in the IC₅₀ value over compound 12. It revealed that 2-aroylquinolines skeleton with an amino group at the R5 position and a methoxy group at the R6 position would abundantly increase the inhibition of proliferation.

N1-substituted quaternary derivatives (compounds 16 and 17) were synthesized using compound 9 as raw material, wherein compound 16 reduced the activity by>10-fold magnitude compared with compound 9 (compound 16 vs compound 9), but compound 17 resulted in drastic loss of activity (compound 17 vs compound 9), thus revealing that the quaternary salts of alkylquinoline and quinoline N-oxide were not preferred.

(B) Tnhibition of Tubulin Polymerization and Colchicine Binding Activity

To examine whether aroylquinolines or aroylquinazoline were microtubule inhibitors acting through the colchicine-binding site, the 2-aroylquinoline (compounds 5, 12 and 15), 6-aroylquinoline (compounds 9 and 14) and reference compounds (compounds 1 and 3) were evaluated for antitubulin activity and the ability to compete for the colchicine-binding site. As shown in Table 3, compounds 9, 12 and 15 were effective in inhibiting microtubule assembly with IC₅₀ values of 2.9, 3.5, and 1.6 μM), respectively. Compound 15 showed more potent antitubulin activity as compared to compound 1 (IC₅₀=2.1 μM) and compound 3 (IC₅₀=4.2 μM), which positively correlated with its antiproliferative activity. Unexpectedly, the moderate cytotoxic compounds 5 and 14 did not inhibit microtubule assembly up to 10 μM. Results of the [³H]-colchicine binding assay indicated that compound 15 was strongly bound to the colchicine-binding domain of tubule with binding affmity comparable to compound 1.

According to the above-mentioned compounds and the preparation methods for the same, the present invention not only provides the antitubulin activity to achieve the antiproliferative activity against cancer cells, but also makes a breakthrough on the research field of anticancer drug. The present invention also provides the relevant synthetic methods to synthesize the compounds of the present invention at the preferred conditions and the reaction reagents.

The above objectives and advantages of the present invention will become more readily apparent to those ordinarily skilled in the art after reviewing the following detailed descriptions and accompanying drawings, in which:

### BRIEF DESCRIPTION OF THE DRAWINGS

FIGS. 1(a) to 1(c) depict (a) the structures of combretastatin A-4 (compound 1 with R═OH) and combretastatin A-4P (CA4P, compound 2 with R═OPO₃Na₂), (b) colchicine, and (c) ABT-751 (compound 4);

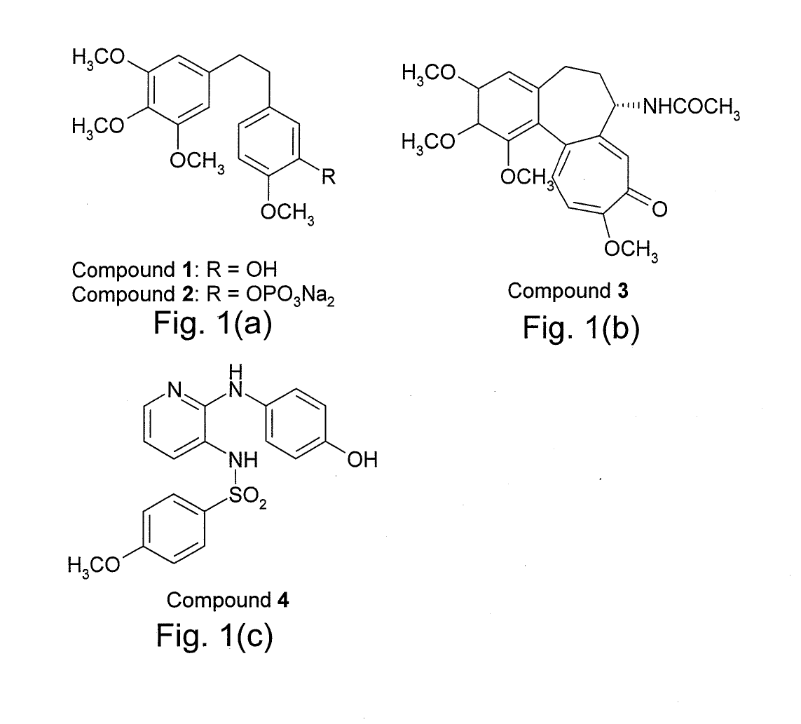

FIGS. 2(a) and 2(b) depict (a) the mechanism for preparing compounds 31, 48, 52, 63 and (b) the mechanism for preparing compounds 71 to 73; and

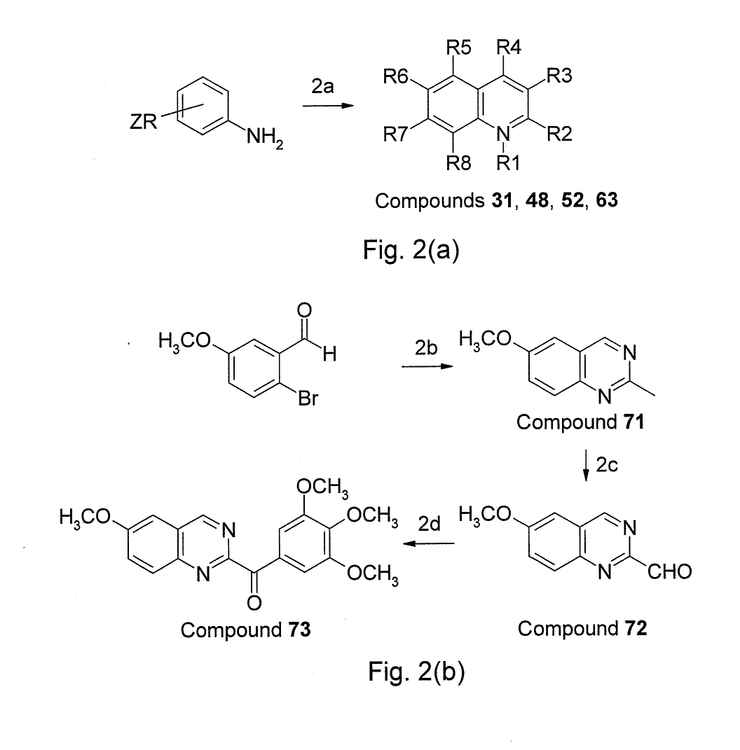

FIGS. 3(a) and 3(b) depict (a) the mechanism for preparing compounds 5 to 11 and (b) the mechanism for preparing compounds 9, 16, 17.

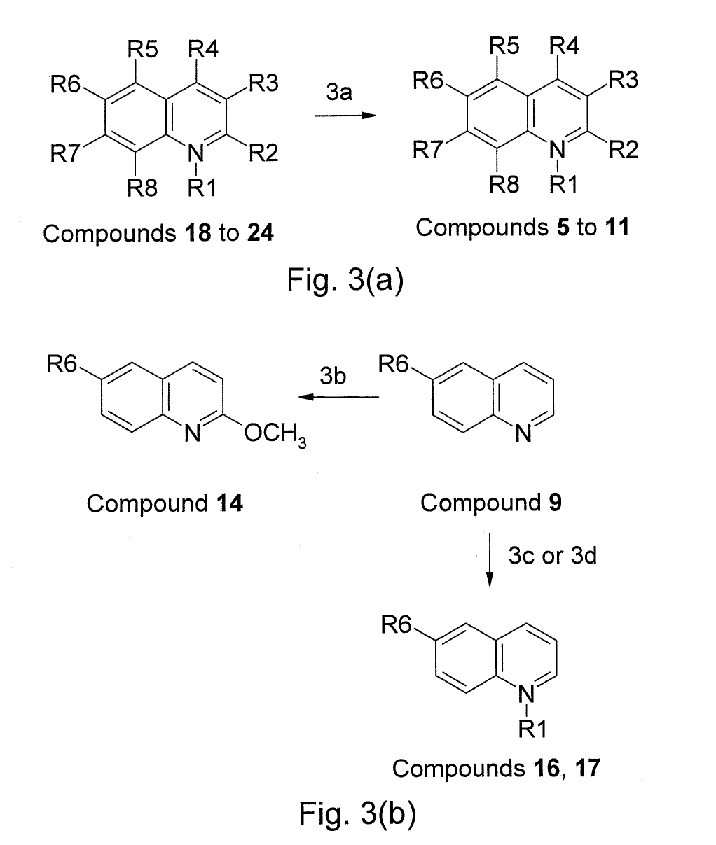

### DETAILED DESCRIPTION OF THE PREFERRED EMBODIMENT

The present invention will now be described more specifically with reference to the following Embodiments. It is to be noted that the following descriptions of preferred Embodiments of this invention are presented herein for purpose of illustration and description only; it is not intended to be exhaustive or to be limited to the precise form disclosed.

### Embodiment 1

### Preparation of Compounds 9 (6-(3′,4′,5′-trimethoxy-benzoyl)quinoline) and 75 (6-(3′,4′,5′-trimethoxybenzoyl)quinoxaline)

A solutuion of 3,4,5,-trimethoxyphenylmagnesium bromide (10 mL, 1.0 M in tetrahydrofuran (THF) prepared in advance) was added slowly to the corresponding 6-quinoline-carboxaldehyde (compound 22, 1.57 g, 10 mmol) in THF (10 mL) at 0° C. The reaction mixture was warmed to room temperature, and stirring was continued for another 1 hour. A saturated ammonium chloride (NH₄Cl) solution was slowly added to hydrolyze the adduct at 0° C. and extracted with ethyl acetate (EtOAc, 15 mL×2) and dichloromethane (CH₂Cl₂, 15 mL×2). The combined organic extract was dried over magnesium sulfate (MgSO₄) and evaporated to give a crude residue, which was dissolved in CH₂Cl₂ (50 mL). Molecular sieves (4 Å, 7.52 g) and pyridinium dichromate (PDC, 7.52 g, 20 mmol) were added to the reaction mixture with stirring at room temperature for 16 hours. The reaction mixture was filtered through a pad of celite. The filtrate was evaporated to give a residue that was purified by silica gel flash column chromatography (EtOAc:n-hexane=2:3) and recrystalized (methanol, CH₃OH) to afford compound 9 (72% yield).

Based on the above-mentioned preparation method, compound 75 (47% yield) was afforded using compound 74 and 3,4,5-trimethoxyphenylmagnesium bromide.

### Embodiment 2

### Preparation of compounds 5 (2-(3′,4′,5′-trimethoxybenzoyl)quinoline), 6 (3-(3′,4′,5′-trimethoxybenzoyl)quinoline), 7 (4-(3′,4′,5′-trimethoxybenzoyl)quinoline), 8 (5-(3′,4′,5′-trimethoxybenzoyl)-quinoline), 10 (7-(3′,4′,5′-trimethoxybenzoyl)quinoline), 11 (8-(3′,4′,5′-trimethoxybenzoyl)quinoline), 12 (6-methoxy-2-(3′,4′,5′-trimethoxybenzoyl)-quinoline), 13 (8-methoxy-4-(3′,4′,5′-trimethoxybenzoyl)quinoline) and 29 (6-methoxy-5-nitro-2-(3′,4′,5′-trimethoxybenzoyl)quinoline)

Based on the preparation method of embodiment 1, a serious of aroylquinoline derivatives bounding 3′,4′,5′-trimethoxybenzoyl substituted groups at R2 to R8 positions of quinoline were synthesized using the raw materials containing carboxaldehyde group. For instance, derivative 5 (64% yield) was afforded from compound 18, derivative 6 (70% yield) was afforded from compound 19, derivative 7 (62% yield) was afforded from compound 20, derivative 8 (66% yield) was afforded from compound 21, derivative 10 (58% yield) was afforded from compound 23, and derivative 11 (57% yield) was afforded from compound 24.

In addition, the preparation method of embodiment 1 also can be adequated in preparing derivative 12 (68% yield) afforded from 6-methoxy-2-quinolinecarboxaldehyde (compound 27), derivative 13 (43% yield) afforded from 8-methoxy-4-quinolinecarboxaldehyde (compound 32), or derivative 29 (57% yield) afforded from 6-methoxy-5-nitro-2-quinolinecarboxaldehyde (compound 28).

### Embodiment 3

### Preparation of compound 14 (2-methoxy-6-(3′,4′,5′-trimethoxybenzoyl)quinoline)

Compound 9 (0.20 g, 0.62 mole) was slowly mixed with CH₂Cl₂ (2 mL) and meta-chloroperoxybenzoic acid (m-CPBA, 0.16 g, 0.93 mmol), and stirring was continued at room temperature for 12 hours. Ten percent (10%) sodium sulfite (Na₂SO₃), the satuarated sodium bicarbonate (NaHCO₃) and the salt solution were sequentially added to the reactive solution and extracted with EtOAc (15 mL×2). The combined organic extract was dried over MgSO₄ and evaporated to be further purified. The residue was dissolved in CH₂Cl₂ (3 mL) and warmed to 50° C. for 12 hours after phosphoryl chloride (POCl₃, 0.6 mL) was added. Solvent was evaporated after the reaction, the adduct then was dissolved in CH₃OH (3 mL) and sodium methoxide (0.12 g, 2.1 mmol) was added to heat at reflux for 3 hours. After extraction with EtOAc (10 mL×3), the combined extracts were basified with sodium bicarbonate (NaHCO₃). The combined organic extract was dried over MgSO₄ and evaporated to give a crude reside that was purified by silica gel column chromatography (EtOAc:n-hexane=3:1) and recrystalized (CH₃OH) to afford compound 14 (51% yield).

### Embodiment 4

### Preparation of compound 15 (5-amino-6-methoxy-2-(3′,4′,5′-trimethoxybenzoyl)quinoline)

Compound 29 (0.2 g, 0.5 mmol) and sodium sulfide nonahydrate (0.87 g, 3.61 mmol) and sodium hydroxide (NaOH, 0.34 g, 8.48 mmol) were stirred with a mixture of ethanol (4 mL) and water (11 mL), and was heated at reflux for 16 hours and placed overnight. Precipitate was harvested using filtration, washed with water, crystallized with methanol, and compound 15 (78% yield) was afforded.

### Embodiment 5

### Preparation of compounds 16 (6-(3′,4′,5′-trimethoxybenzoyl)quinoline N-oxide) and 17 (6-(3′,4′,5′-trimethoxybenzoyl)-1-methylquinolinium iodide)

Compound 9 (0.20 g, 0.62 mol) was slowly mixed with CH₂Cl₂ (2 mL) and m-CPBA (0.16 g, 0.93 mmol), and stirring was continued at room temperature for 16 hours. The adduct was sequentailly washed with 10% Na₂SO₃ and the satuarated NaHCO₃, and extraction was performed on CH₂Cl₂ (20 mL×3). The combined organic extract was dried over MgSO₄ and compound 16 (95% yield) was afforded.

Compound 9 (0.1 g, 0.3 mole) was mixed with iodomethane (CH₃I, 0.1 mL, 1.54 mmol), and stirring was continued at room temperature for 16 hours. After the solvent in the reactive solution was evaporated, compound 17 (91% yield) was afforded.

### Embodiment 6

### Preparation of compound 26 (6-methoxy-2-methyl-5-nitroquinoline)

The 6-methoxy-2-methylquinoline (compound 25, 0.5 g, 2.89 mmol) was added to 65% nitric acid (HNO₃, 2 mL) and 95% sulfuric acid (H₂SO₄, 2 mL) at 0° C. in portion. After stirring for 3 hours, the reaction mixture was quenched and extracted by water and CH₂Cl₂. The organic layers were combined and evaporated to give a residue, which was purified by flash chromatography (EtOAc:n-hexane=1:2) to give compound 26 (75% yield).

### Embodiment 7

### Preparation of compound 27 (6-methoxy-2-quinolinecarboxaldehyde)

To a stirred mixture of selenium dioxide (SeO₂, 3.20 g, 28.86 mmol) and compound 25 (1 g, 5.77 mmol) in p-xylene (20 mL) was heated at reflux for 16 hours. The reaction mixture was filtered through a pad of celite and then evaporated the filtrate to give a residue that was purified by silica gel flash column chromatography (EtOAc:n-hexane=2:3) to afford compound 27 (72% yield).

### Embodiment 8

### Preparation of compound 28 (6-methoxy-5-nitro-2-quinolinecarboxaldehyde)

To a stirred suspension of SeO₂ (2.29 g, 20.6 mmol) and compound 26 (0.9 g, 4.13 mmol) in 1,4-dioxane (40 mL) was heated at reflux for 48 hours. The reaction mixture as filtered through a pad of celite and then evaporated the filtrate to give a residue that was purified by silica gel flash column chromatography (EtOAc:n-hexane=2:3) to afford compound 28 (72% yield).

### Embodiment 9

### Preparation of compound 31 (8-methoxy-4-methylquinoline)

To a stirred solution of o-anisidine (0.92 mL, 8.1 mmol) and ferric chloride (1.3 g, 8.1 mmol) in acetic acid (10 mL), the then methyl vinyl ketone (0.76 mL, 8.9 mmol) was added dropwise over 15 minutes at room temperature. The reaction mixture was heated to 70° C. for one hour followed by the addition of zinc chloride (1.1 g, 8.1 mmol) heating at reflux for another 2 hours. The reaction mixture was cooled, filtered, basified with 10% NaOH solution, extracted with EtOAc (20 mL×3), dried over sodium sulfate (Na₂SO₄) and evaporated to give compound 31 (60% yield).

### Embodiment 10

### Preparation of compounds 32 (8-methoxy-4-quinolinecarboxaldehyde) and 74 (6-quinoxalinecarboxaldehyde)

To a stirred mixture of SeO₂ (0.64 g, 5.77 mmol) and compound 31 (0.2 g, 1.16 mmol) in p-xylene (10 mL) was heated at reflux for 16 hours. The reaction mixture was filtered through a pad of celite and then evaporated the filtrate to give a residue that was purified by silica gel flash column chromatography (EtOAc:n-hexane=2:1) to afford compound 32 (68% yield).

Based on this method, compound 74 (43% yield) was afforded using 6-methylquinoxaline as the raw material.

### Embodiment 11

### Preparation of compound 40 (5-bromo-6-methoxy-2-methyl-quinoline)

Compound 25 (0.3 g, 1.73 mmol) was dissolved in acetonitrile (3 mL) to prepare as a zero-degree-Celsius solution, and N-bromosuccinimide (0.34 g, 1.9 mmol) was added in batch at this temperature within 5 minutes. The brown slurry was warmed to room temperature, and stirring was continued for another 6 hours. The reaction mixture was quenched by adding 10% sodium bisulfite (NaHSO₃, 0.36 mL). Next, the reaction mixture was added to 0.1 N NaOH solution (2.2 mL), and the brown solution (pH 9) was stirred continuously at room temperature for 1 hour and then filtered. The debris was washed with water and evaporated to afford compound 40 (98% yield) as a brown solid.

### Embodiment 12

### Preparation of compounds 41 (5-bromo-6-methoxy-2-quinolinecarboxaldehyde), 46 (5-chloro-6-methoxy-2-quinoline-carboxaldehyde), 72 (6-methoxyquinazoline-2-carbaldehyde) and 86 (5-iodo-6-methoxy-2-quinoline-carboxaldehyde)

To a stirred solution of SeO₂ (0.18 g, 1.59 mmol) in p-xylene (3 mL), and then a solution of compound 40 (0.2 g, 0.79 mmol) in p-xylene (4 mL) was added dropwise at room temperature. The reaction mixture was heated at reflux for 5 hours. The reaction mixture was filtered through a pad of celite and then evaporated the filtrate to give a residue that was purified by silica gel flash column chromatography for (EtOAc:n-hexane=1:2) to afford compound 41 (92% yield).

Based on this method, compound 46 (86% yield) was afforded using compound 45 as raw material, compound 72 (35% yield) was afforded using compound 71, and compound 86 (69% yield) was afforded using compound 85.

### Embodiment 13

### Preparation of compound 42 (5-bromo-6-methoxy-2-(3′,4′,5′-trimethoxybenzoyl)quinoline)

A solution of 3,4,5-trimethoxyphenylmagnesium bromide (5.4 mL, 1.0 M in THF prepared in advance) was added slowly to compound 41 (0.96 g, 3.6 mmol) in THF (5.4 mL) at 0° C. The reaction mixture was warmed to room temperature, and stirring was continued for another 48 hours. A saturated NH₄Cl solution was slowly added to to hydrolyze the adduct at 0° C. and sequentially extracted with EtOAc (15 mL×2) and CH₂Cl₂ (15 mL×2). The combined organic extract was dried over MgSO₄ and evaporated to give a crude residue, which was dissolved in CH₂Cl₂ (50 mL). Molecular sieves (4 Å, 2.7 g) and pyridinium dichromate (7.52 g, 20 mmol) were added to the reaction mixture with stirring at room temperature for 16 hours. The reactive mixture was filtered through a pad of celite. The filtrate was evaporated to give a residue that was purified by silica gel flash column chromatography (EtOAc:n-hexane=1:2) to afford compound 42 (26% yield).

### Embodiment 14

### Preparation of compound 43 (5-cyano-6-methoxy-2-(3′,4′,5′-trimethoxybenzoyl)quinoline)

A mixture of compound 42 (0.20 g, 0.46 mmol) and copper(I) cyanide (CuCN, 0.08 g, 0.93 mmol) was dissolved in dimethyl formamide (DMF, 3 mL) to heat to 120° C. for stirring 17 hours. The reaction mixture was cooled to room temperature, and grounded and mixed with EtOAc. The reaction mixture was filtered/concentrated by silia gel, and the filtrate was purified by silica gel flash column chromatography (EtOAc:n-hexane=1:2) to afford compound 43 (45% yield).

### Embodiment 15

### Preparation of compound 44 (5-(3″-hydroxy-3″-methylbut-1″-ynyl)-6-methoxy-2-(3′,4′,5′-trimethoxybenzoyl)quinoline)

A mixture of compound 42 (0.10 g, 0.23 mmol), tetrakis(triphenylphosphorine)palladium (0.03 g, 0.03 mmol), diisopropylamine (0.42 mL), 1,4-dioxane (2 mL) and 2-methyl-3-butyn-2-ol (0.27 mL, 2.73 mmol) was heated at reflux in nitrogen for 16 hours. After concentrating under reduced pressure, the combined CH₂Cl₂ extract was evaporated to give a residue that was purified by silica gel flash chromatography (EtOAc:n-hexane=2:3) to afford compound 44 (43% yield).

### Embodiment 16

### Preparation of compound 45 (5-chloro-6-methoxy-2-methylquinoline)

A solution of compound 25 (0.3 g, 1.73 mmol) in acetonitrile (3 mL) was cooled to 0° C., and added N-chlorosuccinimide (0.26 g, 1.9 mmol) at this temperature within 5 minutes. The green slurry was continuously stirred at reflux for another 3 hours, and the reaction mixture was quenched by adding 10% Na₂SO₃ (0.36 mL). Next, the reaction mixture was decanted into 0.1 N NaOH solution (2.2 mL), and the slurry (pH 9) was continuously stirred at room temperautere for 1 hour and then filtered. The filtrate was washed and evaparated to afford compound 45 (76% yield) as a brown solid.

### Embodiment 17

### Preparation of compound 48 (5,6,7-trimethoxy-2-quinoline carboxaldehyde)

Crotonaldehyde (2.0 g, 28.6 mL) was added dropwise to a solution of 3,4,5-trimethoxyaniline (5.0 g, 27.3 mmol) in 6 N hydrochloride (HCl, 35 mL) solution. After refluxing for 1 hour, the reaction mixture was cooled to room temperature, and refluxing was continued in cold water for 4 hours with adding zinc chloride (ZnCl₂, 3.72 g, 27.3 mmol). The dark sticky oil was extracted with NaHCO₃ and CH₂Cl₂, and the combined organic extract was dried over MgSO₄ and concentrated under reduced pressure. The debris was dissolved in p-xylene (89 mL), added SeO₂ (6.1 g, 21.4 mmol) to warm to 90° C.-95° C. overnight. The reaction mixture was filtered through a pad of celite and then evaporated the filtrated to give a residue that was purified by silica gel flash column chromatography (EtOAc:n-hexane=1:5) to afford compound 48 (19% yield).

### Embodiment 18

### Preparation of compounds 34 (5-iodo-6-methoxy-2-(3′,4′,5′-trimethoxybenzoyl)quinoline), 47 (5-chloro-6-methoxy-2-(3′,4′,5′-trimethoxybenzoyl)quinoline), 49 (2-(4′-methoxybenzoyl)-5,6,7-trimethoxy-quinoline), 50 (2-(3′-fluoro-4′-methoxybenzoyl)-5,6,7-trimethoxyquinoline), (2-(4′-fluorobenzoyl)-5,6,7-trimethoxyquinoline), 67 (4-(3′-fluoro-4′-methoxybenzoyl)-6,7,8-trimethoxyquinoline), 68 (4-[4′-(N,N-dimethyl)-benzoyl]-6,7,8-trimethoxyquinoline) and 73 (6-methoxy-2-(3′,4′,5′-trimethoxybenzoyl)quinazoline)

Based on the method in Embodiment 13 for preparing compound 42, compound 47 (65% yield) was afforded using compound 46 as raw material. Compound 49 (52% yield) was afforded using compound 48 as raw material and using 4-methoxy-phenylmagnesium bromide in the Grignard reaction. Compound 50 (47% yield) was afforded using compound 48 as raw material and using 3-fluoro-4-methoxyphenylmagnesium bromide in the abovementioned method. Compound 51 (73% yield) was afforded using compound 48 as raw material and using 4-fluoro-phenylmagnesium bromide in the abovementioned method. Compound 34 (26% yield) was afforded by using 3,4,5-trimethoxyphenyl magnesium bromide in the abovementioned method. Compound 73 (53% yield) was afforded using compound 72 as raw material.

Compound 67 (73% yield) was afforded using compound 53 as raw material and using 3-fluoro-4-methoxyphenylmagnesium bromide in this method. Compound 68 (87% yield) was afforded using 4-(N,N-dimethyl)aniline magnesium bromide.

### Embodiment 19

### Preparation of compound 52 (6,7,8-trimethoxy-4-methylquinoline)

A solution of 3,4,5-trimethoxyaniline (1.0 g, 5.46 mmol) in acetic acid (6.8 mL) solution was added to ferric chloride (0.89 g, 5.46 mmol) in the nitrogen gas, and stirring was continued for another 5 minutes. Methyl vinyl ketone (0.52 mL, 6.0 mmol) was slowly added over 15 minutes, and the reaction mixture was heated at 70° C. for 1 hour, and then anhydrous ZnCl₂ (0.74 g, 5.46 mmol) was added to heat at reflux for another 16 hours. The reaction mixture was cooled, filtered, basified with 10% NaOH solution and extracted with EtOAc (20 mL×3). The combined extract was dried over Na₂SO₄ and evaporated to give compound 52 (55% yield).

### Embodiment 20

### Preparation of compounds 53 (6,7,8-trimethoxyquinoline-4-carboxaldehyde) and 54 (4-(4′-methoxybenzoyl)-6,7,8-trimethoxyquinoline)

Based on the method in Embodiment 7 where compound 27 was afforded from compound 25, compound 53 (83% yield) was afforded using compound 52 as raw material via SeO₂ reaction. Compound 54 was afforded using compound 53 as raw material via the preparation method in Embodiment 1.

### Embodiment 21

### Preparation of compound 55 (6-methoxy-5-nitroquinoline)

6-Methoxyquinoline (1.0 mL, 7.24 mmol) was slowly added to a mixture of 65% HNO₃ (4 mL) and 95% H₂SO₄ (4 mL) at 0° C. The reaction mixture was quenched after one-hour stirring, and extracted with CH₂Cl₂ and water. The combined organic extract was evaporated to give a residue, which was purified by silica gel flash column chromatography (EtOAc:n-hexane=1:1) to afford compound 55 (95% yield).

### Embodiment 22

### Preparation of compound 56 (2-chloro-6-methoxy-5-nitroquinoline)

Compound 55 (1.40 g, 6.86 mmol) was slowly mixed with CH₂Cl₂ (22 mL) and m-CPBA (1.77 g, 10.3 mmol) at 0° C., and stirring was continued overnight at room temperature. The reaction mixture was sequentially washed with 10% Na₂SO₃, the saturated NaHCO₃ and the saturated salt solution, and the debris was dissolved in CH₂Cl₂ (33 mL) to heat at reflux overnight with addition of POCl₃ (6.4 mL). The debris was concentrated with reduced pressure and extracted with CH₂Cl₂. The combined organic extract was evaporated to give a residue that was purified by silica gel flash column chromatography (EtOAc:n-hexane=1:3) to afford compound 56 (78% yield).

### Embodiment 23

### Preparation of compound 57 (6-methoxy-5-nitro-2-(3′,4′,5′-trimethoxyphenyl)quinoline)

A mixture of compound 56 (1.0 g, 4.2 mmol), tetrakis(triphenylphosphorine)palladium (0.40 g, 0.36 mmol), 3,4,5-trimethoxyphenylboronic acid (2.70 g, 12.6 mmol), 2 M potassium carbonate (11.5 mL), toluene (120 mL) and ethanol (58 mL) was heated at reflux in the nitrogen gas for 16 hours. The reaction mixture was concentrated with reduced pressure, and the debris was extracted with CH₂Cl₂. The combined organic extract was evaporated to give a residue that was purified by silica gel flash column chromatography (EtOAc:n-hexane=1:2) and recrystalized to afford compound 57 (47% yield).

### Embodiment 24

### Preparation of compounds 58 (5-amino-6-methoxy-2-(3′,4′,5′-trimethoxyphenyl)quinoline), 60 (5-amino-6-methoxy-2-(3′,4′,5′-trimethoxyphenoxy)quinoline), 62 (5-amino-6-methoxy-2-(3′,4′,5′-trimethoxyphenylamino)quinoline), 82 (5-amino-6-methoxy-2-(3′,4′,5′-trimethoxyphenylthio)quinoline) and 84 (5-amino-6-methoxy-2-(3′,4′,5′-trimethoxyphenylsulfonyl)quinoline)

To a solution of compound 57 (0.10 g, 0.27 mmol) in isopropanol (2.7 mL) and water (0.68 mL) was mixed with iron powder (0.05 g, 0.81 mmol) and NH₄Cl (0.06 g, 0.54 mmol) to heat at reflux for 3 hours. The reaction mixture was cooled to room temperature and filtered through a pad of celite. The filtrate was evaporated to extract with EtOAc (20 mL×3). The combined extract was dried over anhydrous MgSO₄ to concentrate under reduced pressure as a brown solid that was purified by silica gel flash column chromatography (EtOAc:n-hexane=1:1) to afford a white compound 58 (80% yield).

Base on the above-mentioned method, compound 60 (80% yield) was afforded by using compound 59 as raw material and mixing iron powder and NH₄Cl in the reaction. Compound 62 (68% yield) was afforded using compound 61. Compound 82 (71% yield) was afforded using compound 81. Compound 84 (76% yield) was afforded using compound 83.

### Embodiment 25

### Preparation of compounds 59 (6-methoxy-5-nitro-2-(3′,4′,5′-trimethoxyphenoxy)quinoline) and 81 (6-methoxy-5-nitro-2-(3′,4′,5′-trimethoxyphenylthio)quinoline)

Based on the method in Embodiment 13 for preparing compound 43, compound 59 (45% yield) was afforded by reacting 3′,4′,5′-trimethoxy phenol, being the raw material, with 2-chloro-6-methoxy-5-nitroquinoline. Compound 81 (63% yield) was afforded by using 3′,4′,5′-trimethoxy benzenethiol as raw material in the reaction.

### Embodiment 26

### Preparation of compound 61 (6-methoxy-5-nitro-2-(3′,4′,5′-trimethoxyphenylamino)quinoline)

3,4,5-Trimethoxyaniline (0.12 g, 0.63 mmol) and compound 56 (0.1 g, 0.42 mmol) were heated to 200° C., and stirring was continued for 10 minutes. The reaction mixture was extracted with CH₂Cl₂ and NaHCO₃. The combined organic extract was evaporated to give a residue that was purified by silica gel flash column chromatography (EtOAc:n-hexane=2:3) to afford a white compound 61 (19% yield).

### Embodiment 27

### Preparation of compound 63 (2-chloro-5,6,7-trimethoxyquinoline)

A mixture of ferrous sulfate (FeSO₄, 4.60 g, 16.37 mmol), 3′,4′,5′-trimethoxyaniline (1.0 g, 5.46 mmol), glycerol (6.5 mL, 88.42 mmol), the concentrated H₂SO₄ (4.4 mL), nitrobenzene (4.1 mL) and glacial acetic acid (4.9 mL) in a round bottom flask was heated to 145° C. for reacting for 6 hours, and then ice water was added. After the distillation, the dark creamy oil was extracted with NaHCO₃ and CH₂Cl₂. The combined organic extract was dried over anhydrous MgSO₄ and concentrated under reduced pressure. The debris was dissolved in CH₂Cl₂ (9 mL) at room temperature with addition of m-CPBA (0.99 g, 5.75 mmol) for overnight. The reaction mixture was washed with 10% Na₂SO₃, the saturated NaHCO₃ and the saturated salt solution. The debris was dissolved in CH₂Cl₂ (13.8 mL), and POCl₃ (2.6 mL) was added at reflux overnight. The reaction mixture was concentrated under reduced pressure, and the debris was extracted with CH₂Cl₂. The combined organic extract was evaporated to give a residue that was purified by silica gel flash column chromatography (EtOAc:n-hexane=1:7) to afford compound 63 (11% yield).

### Embodiment 28

### Preparation of compounds 64 (2-(4′-methoxy-phenyl)-5,6,7-trimethoxyquinoline), 65 (2-[4′-(N,N-dimethylamino)phenyl]-5,6,7-trimethoxyquinoline) and 66 (2-(3′-fluoro-4′-methoxyphenyl)-5,6,7-trimethoxyquinoline)

A mixture of compound 63 (0.10 g, 0.39 mmol), 4-methoxyphenylboronic acid (0.19 g, 1.18 mmol), tetralds(triphenyl-phosphine)palladium (0.04 g, 0.04 mmol), 2 M potassium dichromate (1.1 mL) and toluene (3 mL) in a 10 mL sealed glass flask which had a stir bar therein in advance was disposed in the microwave to react at 160° C. for 10 minutes. The reaction mixture was cooled to room temperature, decanted into water and extracted with EtOAc and NaHCO₃. The collected extract was concentrated under reduced pressure to give a residue that was purified by silica gel flash column chromatography (EtOAc:n-hexane=1:4) to afford a while compound 64 (65% yield).

Based on the above method, compound 65 (68% yield) was afforded by using compound 63, being the raw material, in reaction with 4-(dimethylamino)phenylboronic acid. Compound 66 (36% yield) was afforded using 3-fluoro-4-methyoxyphenylboronic acid.

### Embodiment 29

### Preparation of compounds 30 (5-(4″-hydroxyphenyl)-6-methoxy-2-(3′,4′,5′-trimethoxybenzoyl)quinoline) and 33 (6-methoxy-5-pyridinyl-2-(3′,4′,5′-trimethoxybenzoyl)quinoline)

A mixture of compound 42 (0.10 g, 0.23 mmol), 4-hydroxyphenylboronic acid (0.19 g, 1.18 mmol), tetrakis(triphenylphosphine)-palladium (0.02 g, 0.02 mmol), 2 M potassium dichromate (0.64 mL) and toluene (3 mL) in a 10-mL sealed glass flask which had a stir bar therein in advance was disposed in the microwave to react at 180° C. for 10 minutes. The reaction mixture was cooled to room temperature, decanted into water and extracted with EtOAc and NaHCO₃. The collected extract was concentrated under reduced pressure to give a residue that was purified by silica gel flash column chromatography (EtOAc:n-hexane=1:4) to afford a while compound 30 (78% yield).

Based on the above method, compound 33 (76% yield) was afforded by using pyridine-4-boronic acid as raw material in the reaction.

### Embodiment 30

### Preparation of compound 71 (6-methoxy-2-methylquinazoline)

A mixture of 2-bromo-5-methoxybenaldehyde (0.50 g, 2.33 mmol), acetamidine hydrochloride (0.25 g, 2.56 mmol), L-proline (0.05 g, 0.47 mmol), cesium carbonate (2.28 g, 6.98 mmol) and cuprous iodide (0.05 g, 0.23 mmol) in DMF (20 mL) was heated to 110° C. for stirring for 18 hours. The reaction mixture was cooled and filtered through a pad of celite. The filtrate was evaporated under reduced pressure, and then extracted with EtOAc (20 mL×3). The combined extract was dried over MgSO₄ and evaporated to give a residue that was purified by silica gel flash column chromatography (EtOAc:n-hexane=1:1) to afford compound 71 (41% yield).

### Embodiment 31

### Preparation of compound 83 (6-methoxy-5-nitro-2-(3′,4′,5′-trimethoxyphenylsulfonyl)quinoline)

Compound 81 (0.50 g, 1.25 mmol), CH₂Cl₂ (100 mL) and m-CPBA (0.65 g, 3.75 mmol) were slowly mixed at 0° C. and then warmed to room temperature, and stirred was continued overnight. The reaction mixture was sequentially washed with 10% Na₂SO₃, the saturated NaHCO₃ and the saturated salt solution. The combined organic extract was evaporated to give a residue that was purified by silica gel flash column chromatography (EtOAc:n-hexane=1:1) to afford compound 83 (78% yield).

### Embodiment 32

### Preparation of compound 85 (5-iodo-6-methoxy-2-methyl-quinoline)

Compound 25 (0.30 g, 1.73 mmol) was dissolved in H₂SO₄ (1.8 mL) and cooled to 0° C., and then N-iodosuccinimide (0.80 g, 1.9 mmol) was slowly added at 0° C. during 5 minutes. The reaction mixture was warmed to room temperature, and stirring was continued for 5 minutes. The reaction mixture was quenched by adding ice water. The reaction mixture was decanted into 0.1 N NaOH, and the slurry-like solution (pH 9) was stirred continuously at room temperature for 1 hour and then filtered. The filtrate was washed with water and evaporated to afford compound 40 (98% yield) as a brown solid. Compound 40 was sequentially extracted with EtOAc (15 mL×2) and CH₂Cl₂ (15 mL×2). The combined extract was evaporated to give a residue that was purified by silica gel flash column chromatography (EtOAc:n-hexane=1:3) to afford compound 85 (96% yield).

### Embodiment 33

### Preparation of compound 87 (5-hydroxy-6-methoxy-2-(4′-hydroxy-3′,5′-dimethoxybenzoyl)quinoline)

A solution of compound 15 (0.10 g, 0.03 mmol) in ice water (0.9 mL) and the concentrated H₂SO₄ (0.44 mL) was added dropwise to a solution of sodium nitrite (NaNO₂, 0.03 g, 0.4 mmol) in the water (0.05 mL). The diazonium salt solution was slowly added dropwise to the boiling 6 M H₂SO₄ (1.5 mL), and the reaction mixture was quenched by adding water. The reaction mixture was extracted with EtOAc and water, and the organic extract was evaoprated to give a residue that was purified by silica gel flash column chromatography (EtOAc:n-hexane=2:3) to afford compound 87 (53% yield).

### Embodiment 34

### Preparation of compound 88 (5-[6methoxy-2-(4′-hydroxy-3′,5′-dimethoxybenzoyl)quinoline] disodium phosphate)

A solution of N-chlorosuccinimide (0.19 g, 1.4 mmol) in anhydrous acetonitrile (7 mL) was heated to 40° C., and stirring was continued for 5 minutes and then the heat source was removed. Dibenzyl phosphite (DBP, 0.39 mL, 1.44 mmol) was added dropwise to the reaction mixture, and stirring was continued at room temperature for 4 hours.

In addition, a mixture of compound 87 (0.1 g, 0.28 mmol), anhydrous acetonitrile (3.5 mL) and N,N-dimethylaminopyridine (0.01 g, 0.04 mmol) were added to a 100-mL dried round bottom flask which had a stir bar therein, the reaction temperature was maintained at 10° C.-20° C., and N,N-diisopropylethylamine (0.25 mL, 1.4 mmol) was added. The reaction mixture was cooled to 0° C., and dibenzyl chlorophosphate was slowly added among 5 to 10 minutes, and stirring was continued at room temperature for 16 hours. The reaction mixture was evaporated using the rotary vaccum evaporator, and toluene (5 mL) was added. The reaction mixture was evaoprated, and another toluene (5 mL) was added. The reaction mixture was extracted with CH₂Cl₂, and the combined organic extract was evaporated to give a residue that was purified by silica gel flash column chromatography (EtOAc:n-hexane=1:1). The obtained eluent was evaporated to dissolve in anhydrous CH₂Cl₂ (2 mL), bromotrimethylsilane (0.05 mL, 0.4 mmol) was added at 0° C. for continuously stirring for 3 hours, and then water (1 mL) was added for stirring another 1 hour. The reaction mixture was washed with EtOAc, the organic extract was lyophilized to obtain a brown solid, which was dissolved in ethanol (1.4 mL). Sodium methoxide (0.03 g) was added to the mixture, and the suspension was continuously stirred for 18 hours. The suspension was evaporated, and the brown oil was dissolved in the water. The mixture was washed with EtOAc and lyophilized to afford compound 88 as a brown solid.

### Embodiment 35

### Preparation of compound 76 (6-methoxy-2-(3′,4′,5′-trimethoxybenzoyl)quinoline-5-carboxamide)

Compound 43 (0.30 g, 0.79 mmol), potassium hydroxide (KOH, 0.22 g, 3.95 mmol) and methanol (4 mL) were added to a sealed tube and mixed. The reaction mixture was warmed to 65° C. for 18 hours, and then added to cooling water (15 mL). The mixture was extracted with EtOAc thrice, and the filtrate was filtered to give a residue that was purified by silica gel flash column chromatography (methanol:CH₂Cl₂=1:49) to afford compound 76 (37% yield).

### Embodiment 36

### Preparation of compounds 35 (5-hydroxy-6-methoxy-2-methylquinoline), 36 (5-(tert-butyl-dimethylsilyloxy)-6-methoxy-2-methylquinoline), 37 (5-(tert-butyl-dimethylsilyloxy)-6-methoxyquinoline-2-carbaldehyde) and 38 (5-hydroxy-6-methoxy-2-(3′,4′,5′-trimethoxybenzoyl)-quinoline)

A mixture of compound 40 (0.13 g, 0.52 mmol), tetrakis(triphenyl-phosphine)palladium (0.02 g, 0.02 mmol), 4,4,5,5-tetramethyl-1,3,2-dioxaborolane (pinacolborane, 0.12 mL, 0.77 mmol), triethylamine (0.21 mL, 1.5 mmol) and 1,4-dioxane (2 mL) was added in a 10-mL glass flask which had a stir bar therein in advance was reacted at 160° C. for 15 minutes in the microwave oven. The reaction mixture was cooled to room temperature, and then decanted to water. The mixture was extracted with EtOAc and NaHCO₃. The collected extract was dried over MgSO₄ and concentrated with reduced pressure, the debris was dissolved in ethanol (1.2 mL). The mixture was added NaOH (0.04 g, 1.04 mmol) and hydroxylamine hydrochloride (0.05 g, 0.78 mmol), and stirring was continued at room temperature for 16 hours. The reaction mixture was decanted to water to extract with EtOAc. The combined organic extract was evaporated and dried over anhydrous MgSO₄, and the residue was purified by silica gel flash column chromatography (EtOAc:n-hexane=2:3) to afford compound 35 (38% yield).

tert-Butylchlorodimethylsilane (1.76 g, 11.42 mmol) was mixed with diisopropylethylamine (1.89 mL, 11.42 mmol), and then a solution of compound 35 (0.54 g, 2.85 mmol) in CH₂Cl₂ (17.2 mL) was added. Stirring was continued at room temperature for 18 hours. The reaction solution was decanted to water to extract with CH₂Cl₂. The collected extract was dried over anhydrous MgSO₄. The dried extract was concentrated with reduced pressure to give a residue that was purified by silica gel column flash chromatography (EtOAc:n-hexane=1:3) to afford compound 36 (87% yield).

Based on the preparation method in Embodiment 8, compound 37 (72% yield) was afforded using compound 36 as raw material.

A solution of 3,4,5-trimethoxyphenyl magnesium bromide (1 mole) in THF (1.6 mL) was prepared at 0° C. and was slowly added dropwise to a solution of compound 37 in THE (2.5 mL). The mixture was warmed to room temperature, and stirring was continued for 16 hours. The saturated NH₄Cl solution was slowly added to the reaction mixture at 0° C. to be hydrolyzed, and then reaction mixture was sequentially extracted with EtOAc (15 mL×2) and CH₂Cl₂ (15 mL×2). The combined extract was dried over MgSO₄ and evaporated to give a crude residue, which was dissolved in CH₂Cl₂ (50 mL). Molecular sieves (4 Å, 0.60 g) and PDC (0.60 g, 1.57 mmol) were added to the reaction mixture with stirring at room temperature for 16 hours. The reaction mixture was filtered through a pad of celite. The filtrate was evaporated to give a residue, which was dissolved in THF (2 mL). Tetra-n-butylammonium fluoride (0.41 mL, 1.0 M in THF) was added to the reaction mixture with stirring at room temperature for 16 hours. The residue was extracted with EtOAc and H₂O. The organic layers were combined and evaporated to give a residue, which was purified by flash chromatography (EtOAc:n-hexane=2:3) to give the compound 38, yield 31%.

### Embodiment 37

### Preparation of compound 89 (5-[6-methoxy-2-(3′,4′,5′-dimethoxybenzoyl)quinoline]disodium phosphate)

N-Chlorosuccinimide (0.09 g, 0.68 mmol) was dissolved in anhydrous acetonitrile (3.4 mL). The reaction mixture was then heated to 40° C. and stirred at this temperature for 5 minutes. The heat source was removed, and DBP (0.19 mL, 0.68 mmol) was added dropwise. The reaction mixture was then stirred for 4 hours at room temperature. In a separate 100 mL dry round-bottom flask, equipped with a stir bar, was charged compound 38 (0.1 g, 0.27 mmol) followed by anhydrous acetonitrile (10 mL) and N,N-dimethylaminopyridine (0.01 g, 0.04 mmol). The temperature of the reaction mixture was maintained between 10° C. and 20° C., and N,N-diisopropylethylamine (0.12 mL, 0.68 mmol) was added. The reaction mixture was then cooled to 0° C., and the dibenzyl chlorophosphate solution was added slowly over a period of 5 to 10 minutes. The reaction mixture was then warmed to room temperature and stirred for 16 hours. The solvent was evaporated completely under reduced pressure using a rotary evaporator, followed by the addition of toluene (5 mL). The solvent (toluene) was evaporated under reduced pressure, and additional toluene (5 mL) was added. The residue was extracted with dichloromethane. The organic layers were combined and evaporated to give a residue, which was purified by flash chromatography (EtOAc:n-hexane=1:1) to give the solids. The solids was dissolved in anhydrous dichlorometane (5 mL) at 0° C. was added bromotrimethylsilane (0.18 mL, 1.4 mmol), and the mixture was stirred for 3 hours. Water (1 mL) was added, the solution was stirred for 1 hour and washed with ethyl acetate, and the aqueous phase was freeze-dried to a brown solid. To a solution of the solid in ethanol (5 mL) was added sodium methoxide (0.09 g, 1.62 mmol), and the suspension was stirred for 18 hours. Solvent was removed in vacuo, and the resulting oil was dissolved in water. The solution was washed with ethyl acetate and then freeze-dried to afford brown solid, yield 53%.

The properties of the synthetic compounds were listed as follows.

Compound 5 (2-(3′,4′,5′-trimethoxybenzoyl)quinoline): mp 158-159° C. ¹H NMR (500 MHz, CDCl₃): δ 3.87 (s, 6H), 3.97 (s, 3H), 7.64 (s, 2H), 7.69−7.66 (m, 1H), 7.81−7.78 (m, 1H), 7.92 (d, J=8.1 Hz, 1H), 8.11 (d, J=8.6 Hz, 1H), 8.20 (d, J=8.3 Hz, 1H), 8.36 (d, J=8.5 Hz, 1H). MS (EI) m/z: 323 (M⁺, 100%). HRMS (EI) for C₁₉H₁₇NO₄ (M⁺): calcd, 323.1157; found, 323.1158.

Compound 6 (3-(3′,4′,5′-trimethoxybenzoyl)quinoline): mp 107-109° C. ¹H NMR (500 MHz, CDCl₃): δ 3.88 (s, 6H), 3.97 (s, 3H), 7.11 (s, 2H), 7.65 (t, J=7.5 Hz, 1H), 7.86 (t, J=7.6 Hz, 1H), 7.94 (d, J=8.1 Hz, 1H), 8.20 (d, J=8.5 Hz, 1H), 8.58 (s, 1H), 9.30 (s, 1H). MS (EI) m/z: 323 (M⁺, 100%). HRMS (EI) for C₁₉H₁₇NO₄ (M⁺): calcd, 323.1158; found, 323.1164.

Compound 7 (4-(3′,4′,5′-trimethoxybenzoyDquinoline): mp 109-110° C. ¹H NMR (500 MHz, CDCl₃): δ 3.78 (s, 6H), 3.94 (s, 3H), 7.09 (s, 2H), 7.41 (d, J=4.2 Hz, 1H), 7.55 (t, J=7.6 Hz, 1H), 7.77 (d, J=7.6 Hz, 1H), 7.87 (d, J=8.4 Hz, 1H), 8.20 (d, J=8.4 Hz, 1H), 9.03 (d, J=4.2 Hz, 1H). MS (EI) m/z: 323 (M⁺, 100%). HRMS (EI) for C₁₉H₁₇NO₄ (M⁺): calcd, 323.1158; found, 323.1152.

Compound 8 (5-(3′,4′,5′-trimethoxybenzoyl)quinoline): mp 145-147° C. ¹H NMR (500 MHz, CDCl₃): δ 3.82 (s, 6H), 3.95 (s, 3H), 7.11 (s, 2H), 7.46 (dd, J=4.2, 8.7 Hz, 1H), 7.70 (d, J=6.9 Hz, 1H), 7.76 (t, J=7.7 Hz, 1H), 8.29 (d, J=8.4 Hz, 1H), 8.50 (d, J=8.5 Hz, 1H), 8.98 (d, J=3.2 Hz, 1H). MS (EI) m/z: 323 (M⁺, 100%). HRMS (EI) for C₁₉H₁₇NO₄ (M⁺): calcd, 323.1156; found, 323.1148.

Compound 9 (6-(3′,4′,5′-trimethoxybenzoyl)quinoline): mp 132-134° C. ¹H NMR (500 MHz, CDCl₃): δ 3.87 (s, 6H), 3.96 (s, 3H), 7.11 (s, 2H), 7.51 (dd, J=4.3, 8.2 Hz, 1H), 8.13 (d, J=8.7 Hz, 1H), 8.22 (d, J=8.7 Hz, 1H), 8.26-8.27 (m, 2H), 9.03-9.04 (m, 1H). MS (EI) m/z: 323 (M⁺, 100%). HRMS (EI) for C₁₉H₁₇NO₄ (M⁺): calcd, 323.1158; found, 323.1153.

Compound 10 (7-(3′,4′,5′-trimethoxybenzoyl)quinoline): mp 149-151° C. ¹H NMR (500 MHz, CDCl₃): δ 3.87 (s, 6H), 3.95 (s, 3H), 7.14 (s, 2H), 7.52 (dd, J=4.2, 8.3 Hz, 1H), 7.95 (d, J=8.5 Hz, 1H), 8.00-8.02 (m, 1H), 8.24 (d, J=8.2 Hz, 1H), 8.48 (s, 1H), 9.00-9.01 (m, 1H). MS (EI) m/z: 323 (M⁺, 100%). HRMS (EI) for C₁₉H₁₇NO₄ (M⁺): calcd, 323.1158; found, 323.1166.

Compound 11 (8-(3′,4′,5′-trimethoxybenzoyl)quinoline): mp 153-155° C. ¹H NMR (500 MHz, CDCl₃): δ 3.75 (s, 6H), 3.91 (s, 3H), 7.12 (s, 2H), 7.44 (dd, J=4.1, 8.2 Hz, 1H), 7.63 (t, J=7.5 Hz, 1H), 7.73 (t, J=6.8 Hz, 1H), 7.96 (d, J=8.1 Hz, 1H), 8.22 (d, J=8.1 Hz, 1H), 8.89 (d, J=2.9 Hz, 1H). MS (EI) m/z: 323 (M⁺, 100%). HRMS (El) for C₁₉H₁₇NO₄ (M⁺): calcd, 323.1158; found, 323.1162.

Compound 12 (6-methoxy-2-(3′,4′,5′-trimethoxybenzoyl)quinoline): mp 143-145° C. ¹H NMR (500 MHz, CDCl₃): δ 3.91 (s, 3H), 3.96 (s, 3H), 3.98 (s, 3H), 7.15 (d, J=2.7 Hz, 1H), 7.44 (dd, J=4.0, 9.1 Hz, 1H), 7.64 (s, 2H), 8.06-8.12 (m, 1H), 7.96 (d, J=8.1 Hz, 1H), 8.22 (d, J=8.5 Hz, 1H). MS (EI) m/z: 353 (M⁺, 100%). HRMS (EI) for C₂₀H₁₉NO₅ (M⁺): calcd, 353.1263; found, 353.1262.

Compound 13 (8-methoxy-4-(3′,4′,5′-trimethoxybenzoyl)quinoline): mp 162.5-164.1° C. ¹H NMR (500 MHz, CDCl₃): δ 3.77 (s, 6H), 3.94 (s, 3H), 4.13 (s, 3H), 7.06 (s, 2H), 7.11 (d, J=7.6 Hz, 1H), 7.38 (d, J=8.4 Hz, 1H), 7.43 (d, J=4.1 Hz, 1H), 7.47 (t, J=8.1 Hz, 1H). MS (EI) m/z: 353 (M⁺, 100%). HRMS (EI) for C₂₀H₁₉NO₅ (M⁺): calcd, 353.1264; found, 353.1268.

Compound 14 (2-methoxy-6-(3′,4′,5′-trimethoxybenzoyl)quinoline): mp 182-183° C. ¹H NMR (500 MHz, CDCl₃): δ 3.87 (s, 6H), 3.96 (s, 3H), 4.06 (s, 3H), 6.82 (d, J=5.3 Hz, 1H), 7.11 (s, 2H), 8.12 (s, 2H), 8.65 (s, 1H), 8.85 (d, J=5.3 Hz, 1H). MS (EI) m/z: 353 (M⁺, 100%). HRMS (EI) for C₂₀H₁₉NO₅ (M⁺): calcd, 353.1263; found, 353.1262.

Compound 15 (5-amino-6-methoxy-2-(3′,4′,5′-trimethoxybenzoyl)-quinoline): mp 184.3-185.2° C. ¹H NMR (500 MHz, CDCl₃): δ 3.91 (s, 6H), 3.96 (s, 3H), 4.03 (s, 3H), 4.34 (br, 2H), 7.51 (d, J=9.2 Hz, 1H), 7.64 (s, 2H), 7.69 (d, J=9.1 Hz, 1H), 8.04 (d, J=8.8 Hz, 1H), 8.30 (d, J=8.8 Hz, 1H). MS (EI) m/z: 368 (M⁺, 100%). HRMS (EI) for C₂₀H₂₀N₂O₅ (M⁺): calcd, 368.1373; found, 368.1374.

Compound 16 (6-(3′,4′,5′-trimethoxybenzoyl)-1-methyl-quinoline N-oxide): mp 175-176° C. ¹H NMR (500 MHz, CDCl₃): δ 3.86 (s, 611), 3.96 (s, 3H), 7.07 (s, 2H), 7.39 (dd, J=6.2, 8.2 Hz, 1H), 7.83 (d, J=8.4 Hz, 1H), 8.11 (d, J=8.3 Hz, 1H), 8.29 (s, 1H), 8.62 (d, J=5.9 Hz, 1H), 8.85 (d, J=8.9 Hz, 1H). MS (EI) m/z: 339 (M⁺, 100%). HRMS (EI) for C₁₉H₁₇NO₅ (M⁺): calcd, 339.1107; found, 339.1106.

Compound 17 (6-(3′,4′,5′-trimethoxybenzoyl)-1-methyl-quinolinium iodide): mp 187-188° C. ¹H NMR (500 MHz, CDCl₃): δ 3.88 (s, 6H), 3.98 (s, 3H), 5.03 (s, 3H), 7.08 (s, 211), 8.25 (dd, J=5.8, 8.3 Hz, 1H), 8.49 (d, J=9.0 Hz, 1H), 8.57-8.54 (m, 1H), 8.59 (s, 1H), 9.06 (d, J=8.4 Hz, 1H), 10.52 (d, J=5.6 Hz, 1H). MS (EI): m/z: 338 (M⁺, 100%). HRMS (EI) for C₂₀H₂₀NO₄⁺(M⁺): calcd, 338.1392; found, 338.1392.

Compound 26 (6-methoxy-2-methyl-5-nitroquinoline): ¹H NMR (500 MHz, CDCl₃): δ 2.72 (s, 3H), 4.05 (s, 3H), 7.39 (d, J=8.8 Hz, 1H), 7.52 (d, J=9.4 Hz, 1H), 7.95 (d, J=8.8 Hz, 1H), 8.15 (d, J=9.4 Hz, 1H).

Compound 27 (6-methoxy-2-quinolinecarboxaldehyde): ¹H NMR (500 MHz, CDCl₃): δ 3.98 (s, 3H), 7.14 (d, J=2.5 Hz, 1H), 7.47 (dd, J=2.5, 9.2 Hz, 1H), 7.8 (d, J=8.4 Hz, 1H), 8.13-8.19 (m, 2H), 10.19 (s, 1H).

Compound 28 (6-methoxy-5-nitro-2-quinolinecarboxaldehyde): ¹H NMR (500 MHz, CDCl₃): δ 4.13 (s, 3H), 7.69 (d, J=9.5 Hz, 1H), 8.11 (d, J=8.1 Hz, 1H), 8.20 (d,J=8.2 Hz, 1H), 8.41 (d, J=9.5 Hz, 1H), 10.17 (s, 1H).

Compound 29 (6-methoxy-5-nitro-2-(3′,4′,5′-trimethoxybenzoyl)-quinoline): ¹H NMR (500 MHz, CDCl₃): δ 3.90 (s, 3H), 3.97 (s, 3H), 4.12 (s, 3H), 7.55 (s, 2H), 7.66 (d, J=9.4, 1H), 8.22 (d, J=8.9 Hz, 1H), 8.26 (d, J=8.9 Hz, 1H), 8.35 (d, J=9.4 Hz, 1H).

Compound 30 (5-(4″-hydroxyphenyl)-6-methoxy-2-(3′,4′,5′-trimethoxybenzoyl)quinoline): mp 201-203° C. ¹H NMR (500 MHz, DMSO): δ 3.79 (s, 3H), 3.82 (s, 6H), 3.86 (s, 3H), 6.90 (d, J=8.4 Hz, 2H), 7.13 (d, J=8.4 Hz, 2H), 7.55 (s, 2H), 7.85 (d, J=9.3 Hz, 1H), 7.94 (d, J=8.9 Hz, 1H), 8.01 (d, J=8.9 Hz, 1H), 8.19 (d, J=9.3 Hz, 1H), 9.60 (s, 1H). MS (EI) m/z: 445 (100%). HRMS (EI) for C₂₆H₂₃NO₆ (M⁺): calcd, 445.1525.; found, 445.1526.

Compound 31 (8-methoxy-4-methylquinoline): ¹H NMR (500 MHz, CDCl₃): δ 2.57 (s, 3H), 3.99 (s, 3H), 6.95 (d, J=7.6 Hz, 1H), 7.15 (d, J=4.1 Hz, 1H), 7.39−7.36 (m, 1H), 7.45 (d, J=8.6 Hz, 1H), 8.70 (d, J 4.2 Hz, 1H).

Compound 32 (8-methoxy-4-quinolinecarboxaldehyde): ¹H NMR (500 MHz, CDCl₃): δ 4.12 (s, 3H), 7.16 (d, J=7.8 Hz, 1H), 7.68−7.64 (m, 1H), 7.83 (d, J=4.1 Hz, 1H), 8.55 (d, J=8.6 Hz, 1H), 9.20 (d, J=4.1 Hz, 1H), 10.53 (s, 1H).

Compound 33 (6-methoxy-5-pyridinyl-2-(3′,4′,5′-trimethoxybenzoyl)quinoline): mp 203-205° C. ¹H NMR (500 MHz, DMSO): δ 3.80 (s, 3H), 3.82 (s, 6H), 3.91 (s, 3H), 7.41 (d, J=5.5 Hz, 2H), 7.55 (s, 2H), 7.91-7.99 (m, 3H), 8.30 (d, J=9.0 Hz, 1H), 8.73 (d, J=5.5 Hz, 2H). MS (EI) m/z: 430 (100%). HRMS (EI) for C₂₅H₂₂N₂O₅ (M⁺): calcd, 430.1529; found, 430.1529.

Compound 34 (5-iodo-6-methoxy-2-(3′,4′,5′-trimethoxybenzoyl)-quinoline): mp 202-204° C. ¹H NMR (500 MHz, CDCl₃): δ 3.90 (s, 611), 3.97 (s, 3H), 4.10 (s, 3H), 7.51 (d, J=9.2 Hz, 1H), 7.60 (s, 2H), 8.13 (d, J=8.8 Hz, 1H), 8.20 (d, J=9.2 Hz, 1H), 8.61 (d, J=8.8 Hz, 1H). MS (EI) m/z: 479 (100%). HRMS (EI) for C₂₀H₁₈INO₅ (M⁺): calcd, 479.0230; found, 479.0229.

Compound 35 (5-hydroxy-6-methoxy-2-methylquinoline): ¹H NMR (500 MHz, CDCl₃): δ 2.70 (s, 3H), 3.94 (s, 3H), 6.54 (s, 1H), 7.22 (d, J=8.5 Hz, 1H), 7.40 (d, J=9.0 Hz, 1H), 7.59 (d, J=9.0 Hz, 1H), 8.39 (d, J=9.0 Hz, 1H).

Compound 36 (5-(tert-butyl-dimethylsilyloxy)-6-methoxy-2-methylquinoline): ¹H NMR (500 MHz, CDCl₃): δ 0.21 (s, 6H), 1.07 (s, 9H), 2.69 (s, 3H), 3.90 (s, 3H), 7.21 (d, J=8.5 Hz, 1H), 7.42 (d, J=9.0 Hz, 1H), 7.64 (d, J=9.5 Hz, 1H), 8.35 (d, J=8.5 Hz, 1H).

Compound 37 (5-(tert-butyl-dimethylsilyloxy)-6-methoxy-quinoline-2-carbaldehyde): ¹H NMR (500 MHz, CDCl₃): δ 0.23 (s, 6H), 1.08 (s, 9H), 3.98 (s, 3H), 7.58 (d, J=9.5 Hz, 1H), 7.91 (d, J=9.0 Hz, 1H), 7.95 (d, J=8.5 Hz, 1H), 8.59 (d, J=8.5 Hz, 1H), 10.17 (s, 1H).

Compound 38 (5-hydroxy-6-methoxy-2-(3′,4′,5′-trimethoxy benzoyl)quinoline): ¹H NMR (500 MHz, CDCl₃): δ 3.90 (s, 6H), 3.96 (s, 3H), 4.07 (s, 3H), 6.08 (s, 1H), 7.54 (d, J=9.5 Hz, 1H), 7.79 (d, J=9.5 Hz, 1H), 8.05 (d, J=9.0 Hz, 1H), 8.66 (d, J=8.5 Hz, 1H).

Compound 40 (5-bromo-6-methoxy-2-methylquinoline): ¹H NMR (500 MHz, CDCl₃): δ 2.73 (s, 3H), 4.03 (s, 3H), 7.33 (d, J=8.7 Hz, 1H), 7.46 (d, J=9.2, Hz, 1H), 8.00 (d, J=9.2 Hz, 1H), 8.40 (d, J=8.7 Hz, 1H).

Compound 41 (5-bromo-6-methoxy-2-quinoline-carboxaldehyde): ¹H NMR (500 MHz, CDCl₃): δ 4.11 (s, 3H), 7.61 (d, J=9.2 Hz, 1H), 8.07 (d, J=8.9 Hz, 1H), 8.25 (d, J=9.2 Hz, 1H), 8.66 (d, J=8.7 Hz, 1H), 10.20 (s, 1H).

Compound 42 (5-bromo-6-methoxy-2-(3′,4′,5′-trimethoxybenzoyl)-quinoline): mp 163-165° C. ¹H NMR (500 MHz, CDCl₃): δ 3.90 (s, 6H), 3.96 (s, 3H), 4.10 (s, 3H), 7.57-7.60 (m, 3H), 8.16-8.20 (m, 2H), 8.70 (d, J=8.9 Hz, 1H). MS (EI) m/z: 431 (M⁺, 40%), 195 (100%). HRMS (EI) for C₂₀H₁₈BrNO₅ (M⁺): calcd, 431.0368; found, 431.0367.

Compound 43 (5-cyano-6-methoxy-2-(3′,4′,5′-trimethoxybenzoyl)-quinoline): mp 191-192° C. ¹H NMR (500 MHz, CDCl₃): δ 3.90 (s, 6H), 3.97 (s, 3H), 4.14 (s, 3H), 7.56 (s, 2H), 7.59 (d, J=9.4 Hz, 1H), 8.25 (d, J=8.7 Hz, 1H), 8.39 (d, J=9.4 Hz, 1H), 8.58 (d, J=8.7 Hz, 1H). MS (EI) m/z: 378 (100%). HRMS (EI) for C₂₀H₁₈BrNO₅ (M⁺): calcd, 378.1216; found, 378.1216.

Compound 44 (5-(3″-hydroxy-3″-methylbut-1″-ynyl)-6-methoxy-2-(3′,4′,5′-trimethoxybenzoyl)quinoline): mp 151-153° C. ¹H NMR (500 MHz, CDCl₃): δ 1.76 (s, 6H), 3.90 (s, 6H), 3.96 (s, 3H), 4.07 (s, 3H), 7.53 (d, J=9.3 Hz, 1H), 7.60 (s, 2H), 8.13-8.16 (m, 2H), 8.65 (d, J=8.7 Hz, 1H). MS (EI) m/z: 435 (100%). HRMS (EI) for C₂₅H₂₅NO₆ (M⁺): calcd, 435.1682; found, 435.1681.

Compound 45 (5-chloro-6-methoxy-2-methylquinoline): ¹H NMR (500 MHz, CDCl₃): δ 2.73 (s, 3H), 4.04 (s, 3H), 7.35 (d, J=8.7 Hz, 1H), 7.49 (d, J=9.3, Hz, 1H), 7.97 (d, J=9.3 Hz, 1H), 8.42 (d, J=8.7 Hz, 1H).

Compound 46 (5-chloro-6-methoxy-2-quinoline-carboxaldehyde): ¹H NMR (500 MHz, CDCl₃): δ 4.10 (s, 3H), 7.62 (d, J=9.3 Hz, 1H), 8.06 (d, J=8.7 Hz, 1H), 8.19 (d, J=9.3 Hz, 1H), 8.64 (d, J=8.8 Hz, 1H), 10.18 (s, 1H).

Compound 47 (5-chloro-6-methoxy-2-(3′,4′,5′-trimethoxybenzoyl)-quinoline): mp 176-177° C. ¹H NMR (500 MHz, CDC1₃): 6 3.90 (s, 6H), 3.96 (s, 3H), 4.10 (s, 3H), 7.60-7.61 (m, 3H), 8.13-8.19 (m, 2H), 8.70 (d, J=8.8 Hz, 1H). MS (EI) m/z: 387 (M⁺, 13%), 334 (100%). HRMS (EI) for C₂₀H₁₈ClNO₅ (M⁺): calcd, 387.0874; found, 387.0873.

Compound 48 (5,6,7-trimethoxy-2-quinoline carboxaldehyde): ¹H NMR (500 MHz, CDCl₃): δ 4.03 (s, 3H), 4.05 (s, 3H), 4.07 (s, 3H), 7.37 (s, 1H), 7.90 (d, J=8.4 Hz, 1H), 8.50 (d, J=8.4 Hz, 1H), 10.17 (s, 11-1H).

Compound 49 (2-(4′-methoxybenzoyl)-5,6,7-trimethoxyquinoline): mp 103-105° C. ¹H NMR (500 MHz, CDCl₃): δ 3.90 (s, 6H), 4.01 (s, 3H), 4.02 (s, 3H), 4.09 (s, 3H), 6.99 (d, J=8.8 Hz, 2H), 7.90 (d, J=8.6 Hz, 1H), 8.22 (d, J=8.8 Hz, 2H), 8.51 (d, J=8.5 Hz, 1H). MS (EI) m/z: 353 (M⁺, 56%), 135 (100%). HRMS (EI) for C₂₀H₁₉NO₅ (M⁺): calcd, 353.1263; found, 353.1.266.

Compound 50 (2-(3′-fluoro-4′-methoxybenzoyl)-5,6,7-trimethoxy-quinoline): mp 137-139° C. ¹H NMR (500 MHz, CDCl₃): δ 3.98 (s, 311), 4.02 (s, 6H), 4.09 (s, 3H), 7.05 (t, J=8.2 Hz, 1H), 7.32 (s, 1H), 7.93 (d, J=8.6 Hz, 1H), 8.06-8.09 (m, 2H), 8.52 (d, J=8.6 Hz, 1H). MS (EI) m/z: 371 (100%). HRMS (EI) for C₂₀H₁₈FNO₅ (M⁺): calcd, 371.1169; found, 371.1170.

Compound 51 (2-(4′-fluorobenzoyl)-5,6,7-trimethoxyquinoline): mp 145-146° C. ¹H NMR (500 MHz, CDCl₃): δ 4.02 (s, 6H), 4.09 (s, 3H), 7.16-7.19 (m, 2H), 7.30 (s, 1H), 7.96 (d, J=8.4 Hz, 111), 8.25-8.28 (m, 2H), 8.53 (d, J=8.4 Hz, 1H). MS (EI) m/z: 341 (100%).

Compound 52 (6,7,8-trimethoxy-4-methylquinoline): ¹H NMR (500 MHz, CDCl₃): δ 2.64 (s, 3H), 3.89 (s, 3H), 3.97 (s, 3H), 4.18 (s, 3H), 6.97 (s, 1H), 7.18 (d, J=4.3 Hz, 1H), 8.68 (d, J=4.4 Hz, 1H).

Compound 53 (6,7,8-trimethoxyquinoline-4-carboxaldehyde): ¹H NMR (500 MHz, CDCl₃): δ 4.05 (s, 3H), 4.06 (s, 3H), 4.16 (s, 3H), 7.70 (d, J=4.3 Hz, 1H), 8.31 (s, 1H), 9.07 (d, J=4.3 Hz, 1H), 10.37 (s, 1H).

Compound 54 (4-(4′-methoxybenzoyl)-6,7,8-trimethoxyquinoline): ¹H NMR (500 MHz, CDCl₃): δ 3.83 (s, 3H), 3.88 (s, 311), 4.04 (s, 3H), 4.18 (s, 3H), 6.94-6.96 (m, 3H), 7.30 (d, J=4.4 Hz, 1H), 7.83 (d, J=8.9 Hz, 2H), 8.88 (d, J=4.4 Hz, 1H). MS (EI) m/z: 353 (100%). HRMS (EI) for C₂₀H₁₉NO₅ (M⁺): calcd, 353.1263; found, 353.1263.

Compound 55 (6-methoxy-5-nitroquinoline): ¹H NMR (500 MHz, CDCl₃): δ 4.08 (s, 3H), 7.54 (dd, J=4.2, 8.4 Hz, 1H), 7.60 (d, J=9.4 Hz, 1H), 8.10 (d, J=8.6 Hz, 1H), 8.30 (d, J=9.4 Hz, 1H), 8.88 (d, J=3.4 Hz, 1H).

Compound 56 (2-chloro-6-methoxy-5-nitroquinoline): ¹H NMR (500 MHz, CDCl₃): δ 4.07 (s, 3H), 7.50 (d, J=8.9 Hz, 1H), 7.60 (d, J=9.4 Hz, 1H), 8.03 (d, J=8.9 Hz, 1H), 8.17 (d, J=9.4 Hz, 1H).

Compound 57 (6-methoxy-5-nitro-2-(3′,4′,5′-trimethoxyphenyl)-quinoline): ¹H NMR (500 MHz, CDCl₃): δ 3.93 (s, 3H), 4.01 (s, 6H), 4.09 (s, 3H), 7.39 (s, 2H), 7.59 (d, J=9.4 Hz, 1H), 7.95 (d, J=9.0 Hz, 1H), 8.14 (d, J=8.9 Hz, 1H), 8.37 (d, J=8.7 Hz, 1H).

Compound 58 (5-amino-6-methoxy-2-(3′,4′,5′-trimethoxyphenyl)-quinoline): mp 222-223° C. ¹H NMR (500 MHz, CDCl₃): δ 3.91 (s, 3H), 4.00 (s, 6H), 4.00 (s, 3H), 4.25 (br, 2H), 7.37 (s, 2H), 7.45 (d, J=9.1 Hz, 1H), 7.66 (d, J=9.0 Hz, 1H), 7.74 (d, J=8.9 Hz, 1H), 8.19 (d, J=8.8 Hz, 1H). MS (EI) m/z: 340 (100%). HRMS (EI) for C₁₉H₂₀N₂O₄ (M⁺): calcd, 340.1423; found, 340.1423.

Compound 59 (6-methoxy-5-nitro-2-(3′,4′,5′-trimethoxyphenoxy)-quinoline): ¹H NMR (500 MHz, CDCl₃): δ 3.81 (s, 6H), 3.83 (s, 3H), 4.03 (s, 3H), 6.48 (s, 2H), 7.20 (d, J=9.1 Hz, 1H), 7.47 (d, J=9.3 Hz, 1H), 7.97 (d, J=9.3 Hz, 1H), 8.05 (d, J=9.1 Hz, 1H).

Compound 60 (5-amino-6-methoxy-2-(3′,4′,5′-trimethoxyphenoxy)-quinoline): mp 209-210° C. ¹H NMR (500 MHz, CDCl₃): δ 3.66 (s, 3H), 3.72 (s, 6H), 3.83 (s, 3H), 5.45 (s, 2H), 6.53 (s, 2H), 6.91 (d, J=9.0 Hz, 1H), 6.97 (d, J=9.1 Hz, 1H), 7.33 (d, J=9.0 Hz, 1H), 8.53 (d, J=9.1 Hz, 1H). MS (EI) m/z: 356 (100%). HRMS (EI) for C₁₉H₂₀N₂O₅ (M⁺): calcd, 356.1372; found, 356.1375.

Compound 61 (6-methoxy-5-nitro-2-(3′,4′,5′-trimethoxyphenyl-amino)quinoline): ¹H NMR (500 MHz, CDCl₃): δ 3.84 (s, 3H), 3.85 (s, 3H), 3.87 (s, 3H), 4.01 (s, 3H), 6.86 (s, 2H), 7.03 (d, J=9.3 Hz, 1H), 7.43 (d, J=9.3 Hz, 1H), 7.85-7.87 (m, 2H).

Compound 62 (5-amino-6-methoxy-2-(3′,4′,5′-trimethoxyphenyl-amino)quinoline): mp 222-223° C. ¹H NMR (500 MHz, DMSO): δ 3.60 (s, 3H), 3.79 (s, 6H), 3.80 (s, 3H), 5.23 (s, 2H), 6.81 (d, J=9.2 Hz, 1H), 6.91 (d, J=8.8 Hz, 1H), 7.24 (d, J=8.8 Hz, 1H), 7.40 (s, 2H), 8.20 (d, J=9.2 Hz, 1H), 9.12 (s, 1H). MS (EI) m/z: 355 (M⁺, 85%), 340 (100%). HRMS (EI) for C₁₉H₂₁N₃O₄ (M⁺): calcd, 355.1532; found, 355.1530.

Compound 63 (2-chloro-5,6,7-trimethoxyquinoline): ¹H NMR (500 MHz, CDCl₃): δ 3.96 (s, 3H), 3.98 (s, 3H), 4.05 (s, 3H), 7.16 (s, 1H), 7.23 (d, J=8.6 Hz, 1H), 8.28 (d, J=8.6 Hz, 1H).

Compound 64 (2-(4′-methoxyphenyl)-5,6,7-trimethoxyquinoline): mp 136-137° C. ¹H NMR (500 MHz, CDCl₃): δ 3.88 (s, 3H), 3.99 (s, 3H), 4.02 (s, 3H), 4.07 (s, 3H), 7.03 (d, J=8.7 Hz, 2H), 7.31 (s, 1H), 7.68 (d, J=8.7 Hz, 1H), 8.08 (d, J=8.7 Hz, 2H), 8.37 (d, J=8.7 Hz, 1H). MS (EI) m/z: 325 (100%). HRMS (EI) for C₁₉H₁₉NO₄ (M⁺): calcd, 325.1314; found, 325.1317.

Compound 65 (2-[4′-(N,N-dimethylamino)phenyl]-5,6,7-trimethoxyquinoline): mp 154-155° C. ¹H NMR (500 MHz, CDCl₃): δ 3.04 (s, 611), 3.98 (s, 3H), 4.02 (s, 3H), 4.07 (s, 3H), 6.83 (d, J=8.7 Hz, 1H), 7.68 (d, J=8.7 Hz, 1H), 8.06 (d, J=8.7 Hz, 1H), 8.32 (d, J=8.7 Hz, 1H). MS (EI) m/z: 338 (100%). HRMS (EI) for C₂₀H₂₂N₂O₃ (M⁺): calcd, 338.1630; found, 338.1629.

Compound 66 (2-(3′-fluoro-4′-methoxyphenyl)-5,6,7-trimethoxy-quinoline): mp 129-130° C. ¹H NMR (500 MHz, CDCl3): δ 3.96 (s, 3H), 3.99 (s, 3H), 4.03 (s, 3H), 4.07 (s, 3H), 7.07 (t, J=8.5 Hz, 1H), 7.29 (s, 1H), 7.66 (d, J=8.7 Hz, 1H), 7.85 (d, J=8.4 Hz, 1H), 7.94 (dd, J=12.6, 1.9 Hz, 1H), 8.38 (d, J=8.7 Hz, 1H). MS (EI) m/z: 343 (100%). HRMS (EI) for C₁₉H₁₈FNO₄ (M⁺): calcd, 343.1220; found, 343.1223.

Compound 67 (4-(3′-fluoro-4′-methoxybenzoyl)-6,7,8-trimethoxy-quinoline): mp 117-118° C. ¹H NMR (500 MHz, DMSO): δ 3.73 (s, 3H), 3.90 (s, 3H), 3.92 (s, 3H), 4.05 (s, 3H), 6.86 (s, 1H), 7.28 (s, 1H), 7.45 (s, 1H), 7.52 (s, 1H), 7.70 (s, 1H), 8.85 (s, 1H). MS (EI) m/z: 353 (100%). HRMS (EI) for C₂₀H₁₉NO₅ (M⁺): calcd, 353.1263; found, 353.1263.

Compound 68 (4-[4′-(N,N-dimethypbenzoyl]-6,7,8-trimethoxy-quinoline): ¹H NMR (500 MHz, CDCl₃): δ 3.09 (s, 6H), 3.83 (s, 3H), 4.04 (s, 3H), 4.18 (s, 3H), 6.65 (d, J=9.0 Hz, 1H), 6.96 (s, 1H), 7.29 (d, J=4.0 Hz, 1H), 7.75 (d, J=9.0 Hz, 1H), 8.87 (d, J=4.5 Hz, 1H).

Compound 71 (6-methoxy-2-methylquinazoline): ¹H NMR (500 MHz, CDCl₃): δ 2.86 (s, 3H), 3.94 (s, 3H), 7.11 (d, J=2.0 Hz, 1H), 7.51-7.53 (m, 1H), 7.86 (d, J=9.0 Hz, 1H), 9.22 (s, 1H).

Compound 72 (6-methoxyquinazoline-2-carbaldehyde): ¹H NMR (500 MHz, CDCl₃): δ 3.96 (s, 3H), 7.14 (d, J=2.5 Hz, 1H), 7.57 (dd, J=8.0, 3.0 Hz, 1H), 7.95 (d, J=9.5 Hz, 1H), 9.21 (s, 1H), 9.30 (s, 1H).

Compound 73 (6-methoxy-2-(3′,4′,5′-trimethoxybenzoyl)-quinazoline): ¹H NMR (500 MHz, DMSO): δ 3.77 (s, 3H), 3.85 (s, 9H), 7.13 (s, 2H), 7.48 (d, J=2.5 Hz, 1H), 7.68 (dd, J=7.8, 2.5 Hz, 1H), 8.00 (d, J=9.5 Hz, 1H), 9.18 (s, 1H).

Compound 74 (6-quinoxaline carboxaldehyde): ¹H NMR (500 MHz, CDCl₃): δ 8.23-8.29 (m, 2H), 8.06 (d, J=1.5 Hz, 1H), 8.97 (s, 1H), 10.28 (s, 2H).

Compound 75 (6-(3′,4′,5′-trimethoxybenzoyl)quinoxaline): mp 149-151° C. ¹H NMR (500 MHz, CDCl₃): δ 3.87 (s, 61-1), 3.96 (s, 3H), 7.14 (s, 2H), 8.20-8.25 (m, 2H), 8.49 (d, J=1.1 Hz, 1H), 8.94-8.95 (m, 2H). MS (EI) m/z: 324 (M⁺, 100%). HRMS (EI) for C₁₈H₁₆N₂O₄ (M⁺): calcd, 324.1110; found, 324.1107.

Compound 81 (6-methoxy-5-nitro-2-(3′,4′,5′-trimethoxyphenyl-thio)quinoline): ¹H NMR. (500 MHz, CDCl₃): δ 3.85 (s, 6H), 3.91 (s, 3H), 4.04 (s, 3H), 6.89 (s, 2H), 7.12 (d, J=9.0 Hz, 1H), 7.51 (d, J=9.5 Hz, 1H), 7.83 (d, J=9.0 Hz, 1H), 8.08 (d, J=9.5 Hz, 1H).

Compound 82 (5-amino-6-methoxy-2-(3′,4′,5′-trimethoxyphenyl-thio)quinoline): ¹H NMR (500 MHz, CDCl₃): δ 3.84 (s, 6H), 3.90 (s, 3H), 3.97 (s, 3H), 6.90 (s, 2H), 6.93 (d, J=9.0 Hz, 1H), 7.39 (d, J=9.0 Hz, 1H), 7.53 (s, 1H), 7.95 (d, J=9.0 Hz, 1H).

Compound 83 (6-methoxy-5-nitro-2-(3′,4′,5′-trimethoxyphenyl-sulfonyl)quinoline): ¹H NMR (500 MHz, CDCl₃): δ 3.87 (s, 3H), 3.91 (s, 6H), 4.10 (s, 3H), 7.32 (s, 2H), 7.67 (d, J=9.5 Hz, 1H), 8.27 (dd, J=9.0, 2.5 Hz, 2H), 8.36 (d, J=9.5 Hz, 1H).

Compound 84 (5-amino-6-methoxy-2-(3′,4′,5′-trimethoxyphenyl-sulfonyl)quinoline): ¹H NMR (500 MHz, CDCl₃): δ 3.86 (s, 3H), 3.90 (s, 6H), 4.00 (s, 3H), 7.35 (s, 2H), 7.51 (d, J=9.0 Hz, 1H), 7.69 (d, J=9.5 Hz, 1H), 8.04 (d, J=9.0 Hz, 1H), 8.32 (d, J=9.0 Hz, 1H).

Compound 85 (5-iodo-6-methoxy-2-methyl-quinoline): ¹H NMR (500 MHz, CDCl₃): δ 2.73 (s, 3H), 4.03 (s, 3H), 7.29 (d, J=8.7 Hz, 1H), 7.39 (d, J=9.2, Hz, 1H), 8.02 (d, J=9.2 Hz, 1H), 8.31 (d, J=8.7 Hz, 1H).

Compound 86 (5-iodo-6-methoxy-2-quinolinecarboxaldehyde): ¹H NMR (500 MHz, CDCl₃): δ 4.10 (s, 3H), 7.55 (d, J=9.3 Hz, 1H), 8.04 (d, J=8.7 Hz, 1H), 8.28 (d, J=9.2 Hz, 1H), 8.59 (d, J=9.0 Hz, 1H), 10.23 (s, 1H).

Compound 87 (5-hydroxy-6-methoxy-2-(4′-hydroxy-3′,5′-dimethoxybenzoyl)quinoline): ¹H NMR (500 MHz, CDCl₃): δ 3.91 (s, 6H), 3.96 (s, 3H), 6.96 (d, J=10.0 Hz, 1H), 7.56 (s, 2H), 7.78 (d, J=8.3 Hz, 1H), 7.89 (d, J=10.0 Hz, 1H), 8.20 (d, J=8.3 Hz, 1H).

Compound 88 (5-[6-methoxy-2-(4′-hydroxy-3′,5′-dimethoxy-benzoyl)quinoline]disodium phosphate): ¹H NMR (500 MHz, D₂O): δ 3.79 (s, 6H), 3.83 (s, 3H), 7.28 (s, 2H), 7.66 (d, J=9.5 Hz, 1H), 7.89 (d, J=8.5 Hz, 1H), 7.98 (d, J=9.0 Hz, 1H), 8.43 (d, J=8.5 Hz, 1H).

Compound 89 (5-[6-methoxy-2-(3′,4′,5′-dimethoxybenzoyl)-quinoline]disodium phosphate): ¹H NMR (500 MHz, D₂O): δ 3.89 (s, 6H), 3.93 (s, 3H), 4.09 (s, 3H), 7.35 (s, 2H), 7.82 (d, J=9.0 Hz, 1H), 7.94 (d, J=9.5 Hz, 1H), 7.99 (d, J=8.5 Hz, 1H), 8.81 (d, J=9.0 Hz, 1H).

### Embodiment 38

### Preparation of compound 11 as Tablets

The following components were weighed respectively, mixed and filled in the tablet machine for preparing tablets.

| | |
|---|---|
| 8-(3′,4′,5′-trimethoxybenzoyl)quinoline (compound 11) | 25 mg |
| Lactose | qs |
| Corn flour | qs |

### Experiment 1

### Biological Evaluation, In Vitro Cell Growth Inhibitory Activity

Human oral epidermoid carcinoma KB cells, non-small-cell lung carcinoma H460 cells, colorectal carcinoma HT29 cells and stomach carcinoma MKN45 cells were maintained in RMPI-1640 medium supplied with 5% fetal bovine serum (FBS). KB-VIN10 cells were maintained in growth medium supplemented with 10 nM vincristine, generated from vincristine-driven selection, and displayed overexpression of P-gp170/MDR. Cells in logarithmic phase were cultured at a density of 5000 cells/mL/well in a 24-well plate. KB-VIN10 cells were cultured in drug-free medium for 3 days prior to use. The cells were exposed to various concentrations of the test drugs for 72 hours. The methylene blue dye assay was used to evaluate the effect of the test compounds on cell growth (Finlay et al., 1984). The IC₅₀ values resulting from 50% inhibition of cell growth was calculated graphically as a comparison with the control. Compounds were examined in at least three independent experiments, and the values shown for these compounds are the mean and standard deviation of these data.

### Experiment 2

### Tubulin Polymerization in Vitro Assay

Turbidimetric assays (Liou et al., 2004; Kuo et al., 2004) of microtubules were performed as described by Bollag et al. 1995. In brief, microtubule-associated protein (MAP)-rich tubulin (from bovine brain, Cytoskeleton, Denver, Col.) has been dissolved in reaction buffer (100 mM PIPES (1,4-piperazinediethanesulfulfonic acid, pH 6.9), 2 mM magnesium chloride (MgCl₂), 1 mM GTP (guanosine triphosphate)) in preparing of 4 mg/mL tubulin solution. Tubulin solution (240 μg MAP-rich tubulin per well) was placed in 96-well microtiter plate in the presence of test compounds or 2% (v/v) DMSO (dimethyl sulfoxide) as vehicle control. The increase in absorbance was measured at 350 nm in a PowerWave X Microplate Reader (Bio-Tek Instruments, Winooski, Vt.) at 37° C. and recorded every 30 seconds for 30 minutes. The area under the curve (AUC) used to determine the concentration that inhibited tubulin polymerization to 50% (IC₅₀). The AUC of the untreated control and 10 μM of colchicine was set to 100% and 0% polymerization, respectively, and the IC₅₀ was calculated by nonlinear regression in at least three experiments.

### Experiment 3

### Tubulin Competition-Binding Scintillation Proximity Assay

This assay was performed as described by Tahir et al., 2003. In brief, 0.08 μM of [³H]colchicine was mixed with the test compound and 0.5 μg special long-chain biotin-labeled tubulin (0.5 μg) and then incubated in 100 μL of reaction buffer (80 mM PIPES, pH 6.8, 1 mM EGTA (ethylene glycol tetraacetic acid), 10% glycerol, 1 mM MgCl₂, and 1 mM GTP) for 2 hours at 37° C. Then 80 μg of Streptavidin-labeled SPA (scintillation proximity assay) beads were added to each reaction mixture. Then the radioactive counts were directly measured by a scintillation counter.

While the invention has been described in terms of what is presently considered to be the most practical and preferred Embodiments, it is to be understood that the invention needs not be limited to the disclosed Embodiments. On the contrary, it is intended to cover various modifications and similar arrangements included within the spirit and scope of the appended claims, which are to be accorded with the broadest interpretation so as to encompass all such modifications and similar structures.

TABLE 1
IC₅₀ values of the compounds on the proliferation of cells

| | Cell type (IC₅₀ nM ± SD^{a}) | | | | |
|---|---|---|---|---|---|
| Compound | KB | H460 | HT29 | MKN45 | KB-VIN10 |
| 5 | 173.6 ± 33.1 | 180 ± 25.5 | 148 ± 17 | 245.5 ± 32.7 | 117.3 ± 23.7 |
| 6 | 1800 ± 100 | 1600 ± 380 | 1000 ± 210 | 562 ± 42 | 1100 ± 220 |
| 7 | 3500 ± 700 | 3800 ± 520 | 2300 ± 480 | 920 ± 90 | 2200 ± 600 |
| 8 | 2900 ± 400 | 3800 ± 620 | 2800 ± 310 | 1200 ± 150 | 2300 ± 400 |
| 9 | 24 ± 6.1 | 36 ± 5.5 | 24.6 ± 3 | 16.3 ± 4.5 | 21.5 ± 7.7 |
| 10 | >10000 | >10000 | >10000 | >10000 | >10000 |
| 11 | 8100 ± 700 | 5000 ± 920 | 4500 ± 1100 | 3200 ± 700 | 6000 ± 800 |
| 12 | 27.2 ± 9.8 | 61.5 ± 20.5 | 77 ± 5.7 | 150 ± 31.2 | 21.5 ± 0.6 |
| 13 | 155 ± 15.1 | 193 ± 35.3 | 162.5 ± 28.7 | 147 ± 25.4 | 165.2 ± 33 |
| 14 | 300.6 ± 64.4 | 256.5 ± 34.6 | 205.5 ± 4.9 | 138.5 ± 54.4 | 202.3 ± 26.2 |
| 15 | 0.3 ± 0.1 | 0.4 ± 0.3 | 0.4 ± 0.2 | 0.3 ± 0.2 | 0.2 ± 0.1 |
| 16 | 829.5 ± 171.5 | 1100 ± 150 | 872 ± 86.2 | 617.3 ± 85 | 764 ± 111.1 |
| 17 | >10000 | >10000 | >10000 | >10000 | >10000 |
| 3 | 12 ± 1.2 | 20.1 ± 3 | 13.2 ± 2.3 | 12.4 ± 2 | 128 ± 8 |
| 1 | 2.4 ± 0.2 | 2.6 ± 0.3 | 835 ± 54 | 4.9 ± 0.2 | 1.9 ± 0.4 |

^{a}SD: standard deviation. All experiments were independently performed at least three times.

TABLE 2
50% Inhibition concentration (IC₅₀, nM) of the
compounds on the proliferation of KB cells line

| Compound | IC₅₀ |
|---|---|
| 47 | 45 |
| 49 | >1 |
| 30 | >10 |
| 33 | 472 |
| 66 | >10 |
| 44 | 495 |
| 54 | 1300 |
| 43 | 17 |
| 50 | >10 |
| 42 | 108 |
| 84 | <10 |
| 62 | >10 |
| 58 | >10 |
| 67 | 1673 |
| 15 | 31 |
| 82 | <10 |
| 34 | 451 |
| 64 | >10 |
| 87 | 471 |
| 65 | >10 |
| 68 | 226 |

TABLE 3
Inhibition of tubulin polymerization and
colchicines binding by the compounds

| | tubulin^{a} | colchicine binding^{b} (% ± SD) | |
|---|---|---|---|
| Compound | IC₅₀ ± SD (μM) | 1 μM inhibitor | 5 μM inhibitor |
| 5 | >10 | | |
| 9 | 2.9 ± 0.5 | 51 ± 3 | 72 ± 3 |
| 12 | 3.5 ± 0.6 | 40 ± 3 | 68 ± 2 |
| 14 | >10 | | |
| 15 | 1.6 ± 0.2 | 90 ± 0.5 | 97 ± 1 |
| 3 | 4.2 ± 0.6 | | |
| 1 | 2.1 ± 0.3 | 87 ± 1 | 95 ± 2 |

^{a}Inhibition of tubulin polymerization.
^{b}Inhibition of [³H]colchicine binding. Tubulin was at 1 μM; [³H]colchicine was at 5 μM.

What is claimed is:

1. A pharmaceutical composition, comprising a nitro heterocyclic derivative having a formula I: 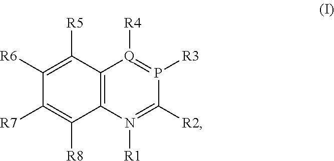
wherein P and Q respectively are ones selected from a group consisting of (i) a first carbon and a second carbon, (ii) a first nitrogen and the second carbon and (iii) the first carbon and a second nitrogen, R1 is a first substituted group being one selected from a group consisting of null, an oxygen, a first C₁-C₈ alkoxy group, a first C₁-C₈ hydrocarbon group and a first C₁-C₈ alkyl halide group, and R2 to R8 respectively are a second substituted group to an eighth substituted group, each of which is one selected from a group consisting of a first hydrogen, a first halide group, a hydroxyl group, a first amino group, a first cyano group, a first nitro group, an aroyl group, a first disodium hydrogen phosphate group, a first diammonium hydrogen phosphate group, a first dipotassium hydrogen phosphate, a first monocalcium phosphate group, a second C₁-C₈ alkoxy group, a C₁-C₈ aromatic group, a second C₁-C₈ hydrocarbon group, a C₁-C₈ alkylthio group, a C₁-C₈ alkyl nitro group, a second C₁-C₈ alkyl halide group, a C₁-C₈ hydroxyl group, a C₁-C₈ aldehyde group, a C₁-C₈ ester group, a C₁-C₈ acidic group, a C₁-C₈ ether group and a C₁-C₈ amide group.

2. The pharmaceutical composition according to claim 1, wherein the first C₁-C₈ alkyl halide group and the second C₁-C₈ alkyl halide group have a second halide group therein being one selected from a group consisting of a fluoride, a chloride, a bromide and an iodide.

3. The pharmaceutical composition according to claim 1, wherein the first C₁-C₈ hydrocarbon group and the second C₁-C₈ hydrocarbon group comprise one of a C₁-C₈ saturated hydrocarbon group and a C₁-C₈ unsaturated hydrocarbon group.

4. The pharmaceutical composition according to claim 3, wherein the C₁-C₈ saturated hydrocarbon group is a C₁-C₈ alkyl group, and the C₁-C₈ unsaturated hydrocarbon group is one of a C₁-C₈ alkenyl group and a C₁-C₈ alkynyl group.

5. The pharmaceutical composition according to claim 1, wherein the first C₁-C₈ hydrocarbon group and the second C₁-C₈ hydrocarbon group form a carbon skeleton having a carbon number ranged between 3 and 5.

6. The pharmaceutical composition according to claim 1, wherein the aroyl group is a ninth substituted group being one selected from a group consisting of an —ArX group, a —CH₂—ArX group, an —O—ArX group, a —CO—ArX group, a —CH₂O—ArX group, a —CO—O—ArX group, an —S—ArX group, an —SO₂—ArX group and an —NH—ArX group, the ArX group is an aromatic group having at least one X group bound thereon, and the at least one X group is a tenth substituted group selected from a group consisting of a second hydrogen group, a third halide group, a second amino group, a second cyano group, a C₁-C₃ alkyl group, a C₁-C₅ hydrocarbon group, a C₁-C₃ alkylthio group, a C₁-C₃ alkyl nitro group, a C₁-C₃ amide group, a C₁-C₃ hydroxyl group, a C₁-C₃ alkyl halide group, a second disodium hydrogen phosphate group, a second diammonium hydrogen phosphate group, a second dipotassium hydrogen phosphate group, a second monocalcimn phosphate group and a combination thereof.

7. The pharmaceutical composition according to claim 1 being used for one selected from a group consisting of treating a cancer, inhibiting a microtubule in a cell and a combination thereof.

8. The pharmaceutical composition according to claim 1 being prepared as a product being one selected from a group consisting of a salt, a solvent, a prodrug, a crystal, a hydrate, a tautomer, a diastereomer, an enantiomer and a metabolite.

9. The pharmaceutical composition according to claim 8, wherein the prodrug is an ester.

10. The pharmaceutical composition according to claim 1 further comprising an additive being one selected from a group consisting of a pharmaceutically acceptable carrier, a dilutent, an excipient and a combination thereof.

11. The pharmaceutical composition according to claim 10, wherein the pharmaceutically acceptable carrier further is a biocapable carrier being one selected from a group consisting of a first solvent, a first dispersing agent, a coating, an antibacterial agent, an antifungal agent and a combination thereof.

12. The pharmaceutical composition according to claim 1 having a dosage form being one selected from a group consisting of a capsule, a tablet, a pill, an emulsion, a liquid suspension, a second dispersing agent and a second solvent.

13. The pharmaceutical composition according to claim 1, wherein the nitro heterocyclic derivative is charged as a first cation, and the pharmaceutical composition is formed as a first salt by adding a first anion with the first cation.

14. The pharmaceutical composition according to claim 13, wherein the anion is an ion being one selected from a group consisting of a chloride ion, a bromide ion, an iodide ion, a sulphate bisulfate ion, a sulfamate ion, a nitrate ion, a phosphate ion, a methanesulfonate ion, a trifluoroacetate ion, a citrate ion, a glutamate ion, a glucuronate ion, a glutarate ion, a malate ion, a maleic ion, a succinate ion, a fumarate ion, a tartrate ion, a tosylate ion, a salicylate ion, a naphthalenesulfonate ion, a lactate ion and an acetate ion.

15. The pharmaceutical composition according to claim 1, wherein the nitro heterocyclic derivative is charged as a second anion, and the pharmaceutical composition is formed as a second salt by adding a second cation with the second anion.

16. The pharmaceutical composition according to claim 15, wherein the second cation is one selected from a group consisting of a sodium ion, a potassium ion, a magnesium ion, a calcium ion and an ammonium cation.

17. The pharmaceutical composition according to claim 15, wherein the second salt further is a quaternary nitrogen salt when the ammonium cation is a tetramethylammonium ion.
